(19) United States
(12) Patent Application Publication
(10) Pub. No.: US 2026/0024630 A1
(43) Pub. Date: Jan. 22, 2026

(54) SYSTEMS AND METHODS OF DEPLOYMENT, SETUP, AND USE OF WORKSPACES USING A CLINICAL STUDY OPERATIONS PLATFORM

(71) Applicant: Trial By Fire Solutions LLC, Oakland, CA (US)

(72) Inventors: Jon Cecchettini, Oakland, CA (US); Nancy Vig, Oakland, CA (US); Steve Pedersen, Oakland, CA (US)

(73) Assignee: Trial By Fire Solutions LLC, Oakland, CA (US)

(21) Appl. No.: 19/274,347

(22) Filed: Jul. 18, 2025

Related U.S. Application Data
(60) Provisional application No. 63/673,624, filed on Jul. 19, 2024.

(51) Int. Cl. G16H 10/20; G06F 21/31

(52) U.S. Cl. CPC G16H 10/20; G06F 21/31

(57) ABSTRACT

An example method comprises receiving, by an on-demand system on a network, different requests to create different, unrelated accounts to manage operations of a first clinical study regarding a first field of study and an unrelated second clinical study regarding an unrelated field of study, creating a first and second account based on credentials provided by the first entity and second entity to manage the operations of the clinical studies, respectively, receiving different configurations to configure different workspaces by the users, and generating dashboards to track and provide operations information and alerts associated to the different entities to manage operations of the first and second clinical studies.

### CROSS-REFERENCE TO RELATED APPLICATIONS

The present application claims priority to and the benefit of U.S. Provisional Patent Application No. 63/673,624, filed Jul. 19, 2024, and entitled “Systems and Methods for the On-Demand and Automated Build of a Study Management and File Management Workspace for Clinical Trials,” which is incorporated by reference herein in its entirety.

### FIELD OF THE INVENTION

Embodiments of the present invention(s) are generally related to the use of a clinical study operation platform for deploying, configuring, and utilizing workspaces for clinical data.

### BACKGROUND

Clinical trials are research studies conducted in humans to evaluate the safety, efficacy, and effectiveness of medical treatments, drugs, devices, or interventions. Clinical trials are a vital part of the process of developing new drugs and are designed to generate data to support regulatory approval, improve medical practice, and advance healthcare. The onboarding process can be tedious and lengthy.

An estimated 500,000 clinical trials are conducted each year worldwide. The time required to establish a platform and/or system for monitoring and managing a clinical trial can vary widely. Typically, software is created for each trial. The time to create and configure the software for clinical trial operations, management, and file storage depends on several factors, including the complexity of the trial, the regulatory environment, the number of sites and participants, and the resources of the sponsoring organization. Clinical trials may take anywhere between 3 to 12 months to onboard, and this can be longer for clinical trials that include multi-site or global clinical studies. The process may include site selection and feasibility, contract negotiation and budget approvals, ethics committee approval, site initiation, participant recruitment and enrollment, and regulatory submissions. Each of these steps may include factors that impact onboarding.

The various members of the clinical trial typically include sponsors, a principal investigator, a clinical research coordinator, and, often, a government regulator. Sponsors represent a company, organization, or individual funding and overseeing the clinical trial. Responsibilities of sponsors include designing the study protocol and objective, providing funding and resources, submitting data to regulatory authorities for approval, monitoring trial progress and safety, and ensuring regulatory compliance. The principal investigator is the lead researcher at a trial site. Their responsibilities include oversight of the clinical trials conducted at the clinical site, confirmation of participant safety and ethical treatment, and reporting trial data. The clinical research coordinator is responsible for day-to-day clinical trial activities at the trial site. Responsibilities of the clinical research coordinator include recruiting and screening participants, scheduling visits and coordinating the study, ensuring accurate data collection and entry, and managing informed consent and participant queries. Government regulators may work for interested departments of their respective official agencies, such as the U.S. Food and Drug Administration (FDA) or European Medicines Agency (EMA).

Some organizations may outsource clinical services to clinical research organizations (CROs) to conduct clinical trials. CROs may manage and oversee multiple clinical trials for different medical treatments, drugs, or devices provided by different sponsors. These clinical trials may be conducted in clinical sites all over the world.

### SUMMARY

In some aspects, the techniques described herein relate to a method including: receiving, by an on-demand system on a network, a first request to create a first account to manage operations of a first clinical study regarding a first field of study; creating a first account based on credentials provided by a first entity to manage the operations of the first clinical study; configuring a first workspace based on a first study configuration information provided by a first user associated with the first entity to manage operations of the first clinical study; and generating a first dashboard to track and provide alerts associated with the first entity to manage operations of the first clinical study.

In some aspects, the techniques described herein relate to a method, further including: receiving, by the on-demand system on the network, a second request to create a second account to manage operations of a second clinical study regarding a second field of study, the first and second fields of study being different from each other; creating a second account based on credentials provided by a second entity to manage the operations of the second clinical study; configuring a second workspace based on second study configuration information provided by a second user associated with the second entity to manage operations of the second clinical study; and generating a second dashboard to track and provide alerts associated to the second entity to manage operations of the first clinical study, the first user having access to the first dashboard and the first workspace based on first user credentials and the second user having access to the second dashboard and the second workspace based on second user credentials, the first user not having access to the second dashboard and the second user not having access to the first dashboard.

In some aspects, the techniques described herein relate to a method, wherein the on-demand system is a multi-tenant system and the first and second entities are different tenants.

In some aspects, the techniques described herein relate to a method, where the first workspace and data associated with the first clinical study is isolated from the second workspace and data associated with the second clinical study.

In some aspects, the techniques described herein relate to a method, further including: receiving, from the first user, a selection of a first reference model from a list of predefined reference models to configure the first workspace, the first reference model being predefined to identify workflows, definitions, and document relationships to assist in managing the first clinical study; and receiving, from the second user, a selection of a second reference model from the list of predefined reference models to configure the second workspace, the second reference model being predefined to identify workflows, definitions, and document relationships to assist in managing the second clinical study.

In some aspects, the techniques described herein relate to a method, wherein the on-demand system does not allow customized source code from the first user, the on-demand system enabling configuration of the first workspace based on predefined functions that may be selected and configured by the first user.

In some aspects, the techniques described herein relate to a method, further including generating a first sandbox with example data to assist in training at least one user associated with the first entity to configure and manage the first workspace for operations management of the first clinical study.

In some aspects, the techniques described herein relate to a method, wherein an authorized user associated with the first entity configures the first workspace by performing one or more of defining categories and subcategories for documents, outlining metadata fields for indexing and searching, specifying document relationships, specifying workflow processes, and establishing rules to ensure compliance with regulations.

In some aspects, the techniques described herein relate to a method, further including: identifying, by the on-demand system, one or more legal documents required based on a type of the first clinical study and geographic locations indicated by the first user; providing, by the on-demand system, the one or more legal documents; and and requiring the one or more legal documents to be effectively signed.

In some aspects, the techniques described herein relate to a method, further including: generating a portfolio workspace, the portfolio workspace allowing the first user to manage and organize data across multiple clinical trials from a workspace interface to perform at least one of tracking, managing, or reporting on aspects of the first clinical study or a third clinical study.

In some aspects, the techniques described herein relate to a method, further including: the first user providing a configuration of a third clinical study; and generating the third clinical study based on a previously created fourth clinical study, the previously created fourth clinical study being previously created by the on-demand system.

In some aspects, the techniques described herein relate to a non-transitory computer-readable medium including executable instructions, the executable instructions being executable by one or more processors to perform a method including: receiving, by an on-demand system on a network, a first request to create a first account to manage operations of a first clinical study regarding a first field of study; creating a first account based on credentials provided by a first entity to manage the operations of the first clinical study; configuring a first workspace based on a first study configuration information provided by a first user associated with the first entity to manage operations of the first clinical study; and generating a first dashboard to track and provide alerts associated with the first entity to manage operations of the first clinical study.

In some aspects, the techniques described herein relate to a non-transitory computer-readable medium, the method further including: receiving, by the on-demand system on the network, a second request to create a second account to manage operations of a second clinical study regarding a second field of study, the first and second fields of study being different from each other; creating a second account based on credentials provided by a second entity to manage the operations of the second clinical study; configuring a second workspace based on second study configuration information provided by a second user associated with the second entity to manage operations of the second clinical study; and generating a second dashboard to track and provide alerts associated to the second entity to manage operations of the first clinical study, the first user having access to the first dashboard and the first workspace based on first user credentials and the second user having access to the second dashboard and the second workspace based on second user credentials, the first user not having access to the second dashboard and the second user not having access to the first dashboard.

In some aspects, the techniques described herein relate to a non-transitory computer-readable medium, the method further including: receiving, from the first user, a selection of a first reference model from a list of predefined reference models to configure the first workspace, the first reference model being predefined to identify workflows, definitions, and document relationships to assist in managing the first clinical study; and receiving, from the second user, a selection of a second reference model from the list of predefined reference models to configure the second workspace, the second reference model being predefined to identify workflows, definitions, and document relationships to assist in managing the second clinical study.

In some aspects, the techniques described herein relate to a non-transitory computer-readable medium, wherein the on-demand system does not allow customized source code from the first user, the on-demand system enabling configuration of the first workspace based on predefined functions that may be selected and configured by the first user.

In some aspects, the techniques described herein relate to a non-transitory computer-readable medium, the method further including generating a first sandbox with example data to assist in training at least one user associated with the first entity to configure and manage a first workspace for operations management of the first clinical study.

In some aspects, the techniques described herein relate to a non-transitory computer-readable medium, wherein an authorized user associated with the first entity configures the first workspace by performing one or more of defining categories and subcategories for documents, outlining metadata fields for indexing and searching, specifying document relationships, specifying workflow processes, and establishing rules to ensure compliance with regulations.

In some aspects, the techniques described herein relate to a system including: at least one processor; and memory containing instructions that are executable by the at least one processor to: receive, by an on-demand system on a network, a first request to create a first account to manage operations of a first clinical study regarding a first field of study; create a first account based on credentials provided by a first entity to manage the operations of the first clinical study; configure a first workspace based on a first study configuration information provided by a first user associated with the first entity to manage operations of the first clinical study; and generate a first dashboard to track and provide alerts associated with the first entity to manage operations of the first clinical study.

In some aspects, the techniques described herein relate to a system, wherein the instructions are further executable by the at least one processor to: receive, by the on-demand system on the network, a second request to create a second account to manage operations of a second clinical study regarding a second field of study, the first and second fields of study being different from each other; create a second account based on credentials provided by a second entity to manage the operations of the second clinical study; configure a second workspace based on second study configuration information provided by a second user associated with the second entity to manage operations of the second clinical study; and generate a second dashboard to track and provide alerts associated to the second entity to manage operations of the first clinical study, the first user having access to the first dashboard and the first workspace based on first user credentials and the second user having access to the second dashboard and the second workspace based on second user credentials, the first user not having access to the second dashboard and the second user not having access to the first dashboard.

In some aspects, the techniques described herein relate to a system, wherein the on-demand system is a multi-tenant system and the first and second entities are different tenants.

### BRIEF DESCRIPTION OF THE DRAWINGS

FIG. 1 depicts a high-level block diagram, including a clinical study operations platform according to some embodiments.

FIG. 2 depicts a diagram of the flow of requests and data between a clinical study operations platform and users according to some embodiments.

FIG. 3 depicts a block diagram of a clinical study operations platform according to some embodiments.

FIG. 4 depicts a flowchart of a method of onboarding and navigation of the clinical study operations platform according to some embodiments.

FIGS. 5A and 5B depict a flowchart of one step of the method of onboarding and navigation of the clinical study operations platform of FIG. 4 according to some embodiments.

FIG. 6 depicts an example user interface from the clinical study operations platform according to some embodiments.

FIG. 7 depicts an example user interface where a user attempts to purchase a subscription to the clinical study operations platform according to some embodiments.

FIG. 8A depicts an example user interface where a user provides payment options using a credit card to provide payment according to some embodiments.

FIG. 8B depicts an example user interface where a user provides payment options using another payment method according to some embodiments.

FIG. 8C depicts a subsequent example user interface where the user provides payment using another payment method and selects the option of electing additional features of the clinical study operations platform according to some embodiments.

FIG. 9 depicts an example user interface that allows a user to navigate to legal documents according to some embodiments.

FIG. 10 depicts an example user interface that provides a subscription receipt according to some embodiments.

FIG. 11 depicts an example user interface of an internal approval to vet the subscription information provided in the example user interfaces of FIG. 7 through FIG. 10 according to some embodiments.

FIG. 12 depicts an example user interface showing details of a subscription according to some embodiments.

FIG. 13A depicts an example user interface showing further details of the subscription according to some embodiments.

FIG. 13B depicts an example user interface of the internal approval process to trigger a new account activation notification according to some embodiments.

FIG. 14 depicts an example of a new account activation notification provided to the subscription according to some embodiments.

FIG. 15 depicts an example user interface from a login user interface according to some embodiments.

FIG. 16A depicts an example user interface of the clinical study operations platform providing a portfolio workspace according to some embodiments.

FIG. 16B depicts an area of the example user interface of FIG. 16A in greater detail according to some embodiments.

FIG. 17 depicts an example user interface, including a form to add a study to the clinical study operations platform according to some embodiments.

FIG. 18A depicts an example user interface, including a study dashboard according to some embodiments.

FIG. 18B depicts an example user interface, including a screen failure table and protocol deviation table for a particular study, according to some embodiments.

FIG. 19 depicts an example user interface, including tasks and milestones associated with a particular study according to some embodiments.

FIG. 20 depicts an example user interface, including a structure to organize, classify, and manage documents for a clinical trial according to some embodiments.

FIG. 21 depicts an example user interface, including a form to invite other users to a particular study according to some embodiments.

FIG. 22 depicts an example compliance portal for users associated with a subscription and other users to access compliance, quality, and validation documents from the clinical study operations platform according to some embodiments.

FIG. 23 depicts an example user interface, including an audit log according to some embodiments.

FIG. 24 depicts a subscription and setting user interface for the clinical study operations platform according to some embodiments.

FIG. 25 depicts a block diagram of an example computing device according to some embodiments.

### DETAILED DESCRIPTION

In various embodiments, a clinical study operations platform may provide an on-demand sign-up model and automated building of portfolio workspaces and study management workspaces. The clinical study operations platform discussed herein may be a multi-tenant system that may include all or some functions of a Clinical Trial Management System (CTMS) and an Electronic Trial Master File (eTMF). The automated build of portfolio workspaces and study management workspaces may generate these workspaces based on configured templates, definitions, and/or properties provided by a user of the clinical study operations platform. In some embodiments, the clinical study operations platform discussed herein is a Commercial Off-The-Shelf (COTS) product intended to support customer-based configurations. In one example, the clinical study operations platform does not support client-customized open source coding (e.g., configurations are available within the parameters of the of the clinical study operations platform such as table creation, predefined menus, and pre-programmed functionality that is readily available to all new clients depending upon subscription and/or clinical study).

The clinical study operations platform may enable maintenance and management of planning, performing, and reporting functions across the areas of contact and organization management, milestones and tasks, forecasting, contracts and payments, screening and enrollment, protocol deviations, monitoring visit calendaring, report, and letter creation.

In some embodiments, the clinical study operations platform may allow automated access for users to access or generate documentation for software validation and regulated electrical systems for managing clinical studies. For example, the clinical study operations platform may provide government agencies such as the FDA as well as the EMA access to the platform, documents, and/or reports (e.g., either previously stored or dynamically generated as needed) for regulation purposes.

Different managers or operators of different, unrelated clinical studies may access the clinical study operations platform discussed herein and create their own instance for tracking, managing, filing, and other aspects of operations management for their particular clinical study. Each instance may be configured by that particular user or operator to enable flexibility, scalability, centralization, and/or efficiency.

In various embodiments, systems and methods described herein transform what was once a slow, expensive, and highly customized process into a fast, flexible, and user-driven experience for managing clinical studies. In some embodiments, unlike traditional systems that require months or even years to set up, some embodiments of systems described herein utilize automated, on-demand setup with default configurations. This allows new clinical study workspaces to be created and ready for use almost immediately. Further, instead of relying on custom programming or vendor intervention for every change, some embodiments described herein enable users to configure and customize their study environments directly through the application. This flexibility dramatically reduces setup time and ongoing maintenance. Moreover, clients may, in some embodiments, self-manage their subscriptions, add or remove services, and scale their usage as their needs change without lengthy negotiations or new contracts for each adjustment.

Some embodiments have a multi-tenant architecture that supports multiple clients on the same infrastructure, enabling efficient resource use and rapid provisioning of new workspaces.

By automating setup and minimizing manual steps, some embodiments described herein eliminate the vendor-imposed delays and bottlenecks common in bespoke systems.

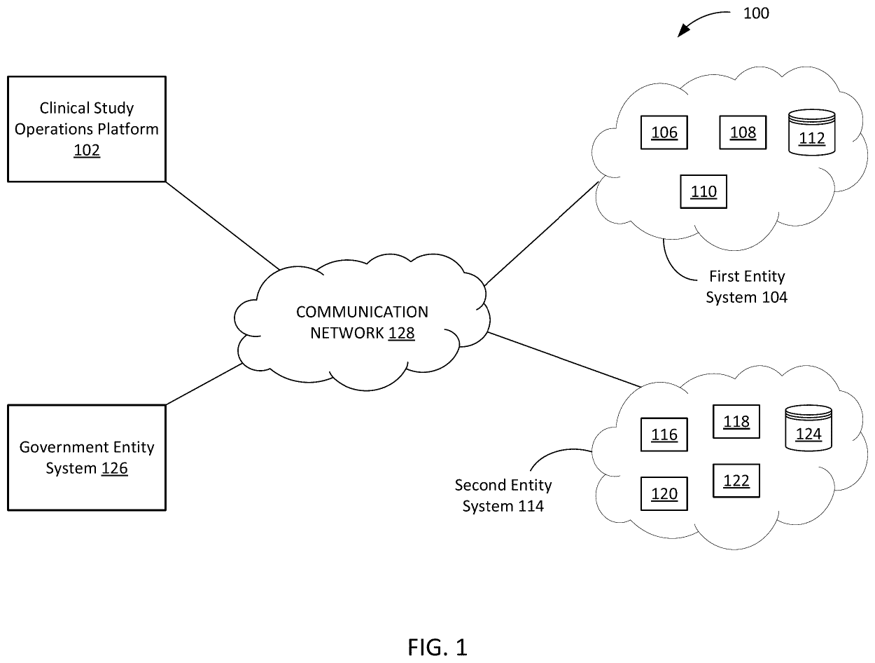

FIG. 1 depicts a high-level block diagram of an environment 100, including a clinical study operations platform 102 according to some embodiments. The environment 100 includes the clinical study operations platform 102, a first entity system 104, and a second entity system 114. In this example, the clinical study operations platform 102 is a product intended to support customer-based configurations, but not specific, bespoke customer programming. As such, configurations (e.g., study requirements and specific functionality) are pre-programmed to be available. In some embodiments, the clinical study operations platform 102 is a multi-tenant system. The multi-tenant system may reduce operational costs, provide for simplified updates and maintenance, and easier onboarding of new tenants. In some examples, by being a multi-tenant system, templates, models, pre-programmed functionality, pre-programmed roles, and pre-programmed configuration options may be extended across tenants.

In this example, the clinical study operations platform 102 assists in the planning, management, and reporting to support clinical studies (e.g., clinical trials). A clinical study may be, for example, a research program. In this example, a clinical study may be a research program to evaluate a new medical drug or device. The data may be used by sponsors to obtain regulatory agency approval. As such, management of operations, configuration of the particular portion of the clinical study operations platform 102, protocol deviations, security, and document retention for audits and compliance assessment may be required. As discussed herein, a clinical study and a clinical trial are used synonymously.

A multi-tenant system is a software architecture where a single instance of an application serves multiple independent users or organizations, known as tenants. Each tenant shares the same application and infrastructure but has its own isolated data and configuration to ensure privacy and security. As such, multiple tenants may utilize the clinical study operations platform 102 to manage unrelated clinical studies.

The clinical study operations platform 102 may maintain and manage planning, performing, and reporting functions across the areas of contact and organization management of the clinical study. The clinical study operations platform 102 may provide support for creating milestones and tasks, forecasting, contracts and payments, screening and enrollment, protocol deviations, and monitoring visit calendaring. In various embodiments, the clinical study operations platform 102 may also provide a formalized means of organizing and storing documents, files and other digital content for clinical studies that may be required for compliance with government regulatory agencies.

The clinical study operations platform 102 may be a multi-tenant system that enables different, unrelated entities (discussed herein) to manage unrelated clinical studies. For example, the clinical study operations platform 102 may provide a single instance to serve multiple, unrelated clients (e.g., unrelated entities) to manage different, unrelated clinical studies. Each tenant or entity may operate in a logically isolated environment, but the underlying infrastructure and applications may be shared among all tenants. The clinical study operations platform 102 may provide operations, management, and document services as a software as a service (SaaS) system. As such, the clinical study operations platform 102 provides significant advantages in scalability, efficiency, and centralized maintenance over the prior art, thereby overcoming technical hurdles and challenges created by the previous technical approach.

The clinical study operations platform 102 may enable different entities with very different clinical studies to create a system to support, manage, and provide documentation related to those particular studies. The clinical study operations platform 102 may be “on-demand.” For example, a user associated with an entity may access the Internet and create an account by purchasing a subscription on the clinical study operations platform 102. The user may then configure the clinical study operations platform 102 to support the particular needs and operations required for their particular clinical study.

The clinical study operations platform 102 may streamline the management of multiple clinical trials, ensuring efficient operations, regulatory compliance, and secure document management. In some embodiments, a user may access the clinical study operations platform 102 to quickly create a clinical study workspace. Unlike traditional systems that require months or even years to set up, the clinical study operations platform 102 may utilize an automated, on-demand setup with default configurations. This allows new clinical study workspaces to be created and ready for use almost immediately. Instead of relying on custom programming or vendor intervention for every change, the clinical study operations platform 102 may enable users to configure and customize their study environments directly through the application. This flexibility dramatically reduces setup time and ongoing maintenance. Further, Clients of the clinical study operations platform 102 may self-manage their subscriptions, add or remove services, and scale their usage as their needs change without lengthy negotiations or new contracts for each adjustment.

In one example, the clinical study operations platform 102 may receive a request from a first user associated with a first entity to review options and subscriptions to create one or more accounts to manage a clinical trial to study a drug lifecycle. The user may access a webpage of the clinical study operations platform 102 (or on another server) on the Internet to review study information, pricing, legal information, onboarding configurations, and the like. The user associated with the first entity may provide credentials and purchase a subscription to enable the management of the drug lifecycle study.

In this example, the user may be a sponsor of a clinical study. As discussed herein, the sponsor is an individual, institution, company, or organization that initiates and takes responsibility for a clinical study. For example, the user (e.g., the sponsor) may develop the study protocol and provide the financial resources required for the trial. The user (e.g., sponsor) may further ensure the study complies with regulatory requirements and obtains necessary approvals from authorities (e.g., the FDA or EMA) and may own the data generated and be responsible for analyzing and reporting the results.

The first user may configure the clinical study operations platform 102 to assist in operations management, digital signing, document retention, deviation alerts, and/or the like. In various embodiments, the first user (e.g., sponsor) may add other users, such as users associated with a contract research organization (CRO) and/or clinical research associate(s) (CRAs). In this example, a CRO is an independent organization contracted by the sponsor to perform one or more trial-related duties and functions such as handling day-to-day operations such as site selection, patient recruitment, and logistics. The CRO (or users associated with the CRO) may assist in preparing and submitting documentation to regulatory bodies as well as implement quality control measures to ensure data integrity and compliance with Good Clinical Practice (GCP). A CRA is a professional responsible for monitoring the progress of clinical trials at investigative sites, ensuring compliance with the study protocol, GCP, and regulatory requirements. They may perform site monitoring, initiation visits, routine monitoring, data verification, training, and support at the site.

In some embodiments, the clinical study operations platform 102 provides a sandbox. A sandbox is an example operations system in the clinical study operations platform 102 with artificial or “dummy” data to enable the new user to interact and learn from the system.

As discussed herein, the clinical study operations platform 102 may be a multi-tenant system configured to allow multiple, unrelated entities to establish a subscription on the clinical study operations platform 102 to manage operations and documents for different, unrelated studies. To further the example immediately above, the clinical study operations platform 102 may receive a request from a second user (e.g., another sponsor) associated with a second entity to review options and subscriptions to create one or more accounts to manage a different clinical trial (e.g., a cancer study). The second user associated with the first entity may provide credentials and purchase a subscription to enable management of the cancer study. The second user may configure the clinical study operations platform 102 to assist in operations management, digital signing, document retention, deviation alerts, and/or the like for this study (their configurations are different than the configurations for the drug study). In some embodiments, the clinical study operations platform 102 provides the same sandbox to both users upon initial account creation. As before, the sandbox is an example operations system in the clinical study operations platform 102 with the same artificial or “dummy” data to enable the new user to interact and learn from the system.

In this example, the clinical study operations platform 102 may allow one instance of software associated with the clinical study operations platform 102 to service multiple tenants. A tenant in this example may be a group of users who share common access with specific privileges to an instance of the clinical study operations platform 102. The use of multi-tenanted systems makes software updates and maintenance simpler and quicker as they are accomplished once on the central instance. This eliminates the requirement for each software instance to be updated individually.

The clinical study operations platform 102 may provide an on-demand, automated clinical study setup that allows a user associated with a subscription to sign up, set up, and build portfolio workspace and/or study workspace. The clinical study operations platform 102 may collect and track workflows for the CTMS. In some embodiments, the CTMS provided by the clinical study operations platform 102 may be validated to meet regulatory standards made by government regulators such as the FDA.

As discussed herein, the first entity system 104 may establish a subscription to the clinical study operations platform 102. The first entity system 104 may be any number of digital devices operated, managed, and/or associated with an entity such as a sponsor, pharmaceutical company, research, or educational facility. Similarly, the second entity system 114 may also establish a subscription to the clinical study platform 102 to manage a different clinical study. The second entity system 114 may be any number of digital devices operated, managed, and/or associated with an entity such as a sponsor, pharmaceutical company, research, or educational facility.

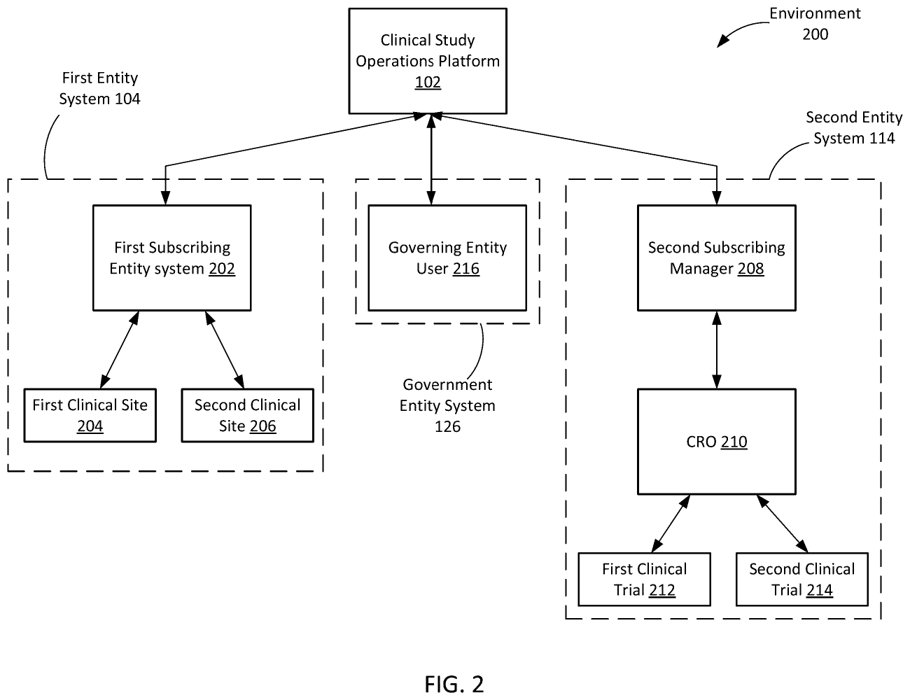

The first entity system 104 may include any number of digital devices, such as, for example, user systems 106, 108, and 110. The user systems 106, 108, and 110 of the first entity system 104 may be utilized by one or more members of the first entity system 104. For example, a first subscribing entity system 202 (see FIG. 2) of the second entity system 114 may represent a computing device utilized by a sponsor of a new drug that is in development and requires an Investigational New Drug (IND) application with the FDA in the United States and clinical trials. A subscribing user may be a user associated with an entity (e.g., sponsor) that establishes the subscription using the clinical study operations platform 102. In various embodiments, the subscribing user may establish or be associated with one or more authorized users. An authorized user is any user that may be authorized to access and/or interact with the clinical study operations platform 102 by virtue, at least in part, based on the subscription (e.g., the subscribing user may, after establishing the subscription, add and/or authorize any number of authorized users to access and interact with the clinical study operations platform 102 for at least one clinical study established and associated with the subscription.

Other user systems 108 and 110 of the first entity system 104 represent computing devices utilized by clinical trials at different clinical trial sites. The first entity system 104 may further include or be linked to any number of clinical datastore(s) 112. Data and documents associated with one or more clinical trials previously executed or currently being executed by the first entity system 104 may be stored in the clinical datastore(s) 112. Data stored in the clinical datastore(s) 112 may include private, confidential participant data associated with the participants of the clinical trials. The clinical datastore(s) 112 may be a cloud-based datastore or an on-premises datastore. In some embodiments, private, confidential participant data associated with the participants of the clinics is not stored by the clinical study operations platform 102. In one example, a subset of the clinical datastore(s) 112 is accessible or provided to the clinical study operations platform 102.

Similar to the first entity system 104, the second entity system 114 may establish a subscription on the clinical study operations platform 102. The clinical study operations platform 102 may allow the software being executed by the clinical study operations platform 102 to service users or even other tenants associated with the second entity system 114. The second entity system 114 may include any number of digital devices, including, for example, user systems 116, 118, 120, and 122. The user systems 116, 118, 120, and 122 of the second entity system 114 may be utilized by one or more members of the second entity system 114. The second entity system 114 may further include clinical datastore(s) 124. Data associated with one or more clinical trials previously executed or currently being executed by the second entity system 114 may be stored in the clinical datastore(s) 124.

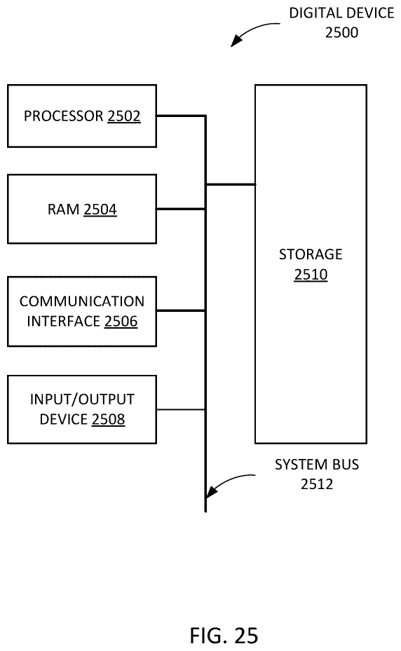

The user systems 106, 108, 110, 116, 118, 120, and 122 facilitate communication between users and other associated systems. In some embodiments, the user systems 106, 108, 110, 116, 118, 120, and 122 may be or include one or more mobile devices (e.g., smartphones, cell phones, smart watches, tablet computer, or the like), desktop computers, laptop computers, and/or the like. Further details about digital devices will be discussed in regard to FIG. 25.

In some embodiments, a communication network 128 represents one or more computer networks (for example, LANs, WANs, and/or the like). The communication network 128 may provide communication between or among any of the clinical study operations platform 102, first entity system 104, and/or the second entity system 114. In some implementations, the communication network 128 comprises computer devices, routers, cables, and/or other network topologies. In some embodiments, the communication network 128 may be wired and/or wireless. In various embodiments, the communication network 128 may comprise the Internet, one or more networks that may be public, private, IP-based, non-IP based, and so forth.

In some embodiments, members of a government agency (e.g., regulators, agents, and/or related contractors) may access the clinical study operations platform 102, such as via a government entity system 126, to view data and/or request one or more report(s) (e.g., for compliance, contract requirements, accuracy, consistency, and/or reliability of clinical data). In some embodiments, data should remain unchanged (unless otherwise authorized) and traceable while maintaining authenticity and quality from data creation to data archival.

FIG. 2 depicts a diagram of the flow of requests and data between a clinical study operations platform and users according to some embodiments. An environment 200 includes various members of different entities, including first entity system 104 and second entity system 114, which utilize the clinical study operations platform 102. Here, the clinical study operations platform 102 is a multi-tenant system.

The first subscribing entity system 202 of the first entity system 104 may purchase a subscription using the clinical study operations platform 102 to monitor, document, and manage operations related to a clinical study. In one example, the first subscribing entity system 202 may be a sponsor of one or more clinical trials required to develop a new drug with the FDA. The first subscribing entity system 202 may purchase or establish a subscription to the clinical study operations platform 102 in order to manage content related to multiple studies, such as calendar events, contact and organizational records, and provide documents and Electronic Visit Report (EVR) templates.

In some embodiments, the first subscribing entity system 202 assesses their clinical study needs and reviews subscription options and legal agreements. Based on the study needs and subscription options, the first subscribing entity system 202 may select and purchase a subscription. Subsequently, the first subscribing entity system 202 may have access to a dedicated platform to create one or more workspaces for one or more clinical studies. The first subscribing entity system 202 may configure each workspace for the needs of the particular clinical study.

In one example, the first subscribing entity system 202 may configure the platform (e.g., their workspace) to define categories and subcategories for documents, outline metadata fields for indexing and searching, specify document relationships, workflow processes, and establishing rules to ensure compliance with regulations. Each function may be pre-programmed and accessible on the platform of the clinical study operations platform 102 to enable the first subscribing entity system 202 to perform these configurations. In some embodiments, the clinical study operations platform 102 provides an option for the first subscribing entity system 202 to select a reference model (e.g., a Drug Information Association (DIA) reference model or Clinical Data Interchange Standards Consortium (CDISC) reference model) from a pull-down menu. Each reference model may include the above configurations that automatically create and configure the workspace as per the model. The first subscribing entity system 202 may then continue to make changes to one or more of the pre-configurations triggered by the reference model.

In various embodiments, the clinical study operations platform 102 may create a clinical study based on another, preexisting clinical study (e.g., by applying and providing initial configurations for the same or different sponsors of similar clinical studies). In some embodiments, functionality of the clinical study operations platform 102 that is not going to be used in monitoring a particular clinical study may be masked by the system for that particular clinical study workspace or for a portfolio workspace (as needed).

The first subscribing entity system 202 may purchase a subscription using the clinical study operations platform 102 to aid in managing operations related to the clinical study at a first clinical site 204 and a second clinical site 206. For example, the clinical study operations platform 102 may assist the site staff in each of the first clinical trial at the first clinical site 204 and the second clinical trial at the second clinical site 206 in calendaring participant visits, screening, enrollment, and documentation. Once the first subscribing entity system 202 completes the onboarding process, the first subscribing entity system 202 can invite other users, such as the site staff of the first clinical trial and the second clinical trial.

The environment 200 includes a second subscribing manager 208. In this example, the second subscribing manager 208 may be a head researcher supervising a series of clinical trials. In this example, the second subscribing manager 208 may optionally engage the services of a clinical research organization (CRO) to manage and oversee a third clinical trial 212 and a fourth clinical trial 214. It will be appreciated that one or more CROs may manage any number of clinical trials for any number of subscribers.

Each of the third clinical trial 212 and the fourth clinical trial 214 may include clinical trials performed at different clinical sites. In some embodiments, members of each of the CRO may be given access to the clinical study operations platform 102 by the second subscribing manager 208. Information regarding the tracking, monitoring, supervisors, milestones, and/or the like for each of the third and fourth clinical trials 212 and 214 may be available on the clinical study operations platform 102 (e.g., on different workspaces within the clinical study operations platform 102, such as a first clinical trial workspace and a second clinical trial workspace, or on a single workspace, such as a portfolio workspace).

In this example, the second subscribing manager 208 may configure the platform to define categories and subcategories for documents, outline metadata fields for indexing and searching, specify document relationships, workflow processes, and ensure compliance with regulations. Each function may be pre-programmed and accessible on the platform of the clinical study operations platform 102 to enable the second subscribing manager 208 to perform these configurations. In some embodiments, the clinical study operations platform 102 provides an option for the second subscribing manager 208 to select a reference model from a pull-down menu. Each reference model may include the above configurations that automatically create and configure the workspace as per the model. The second subscribing manager 208 may then continue to make changes to one or more of the pre-configurations triggered by the reference model.

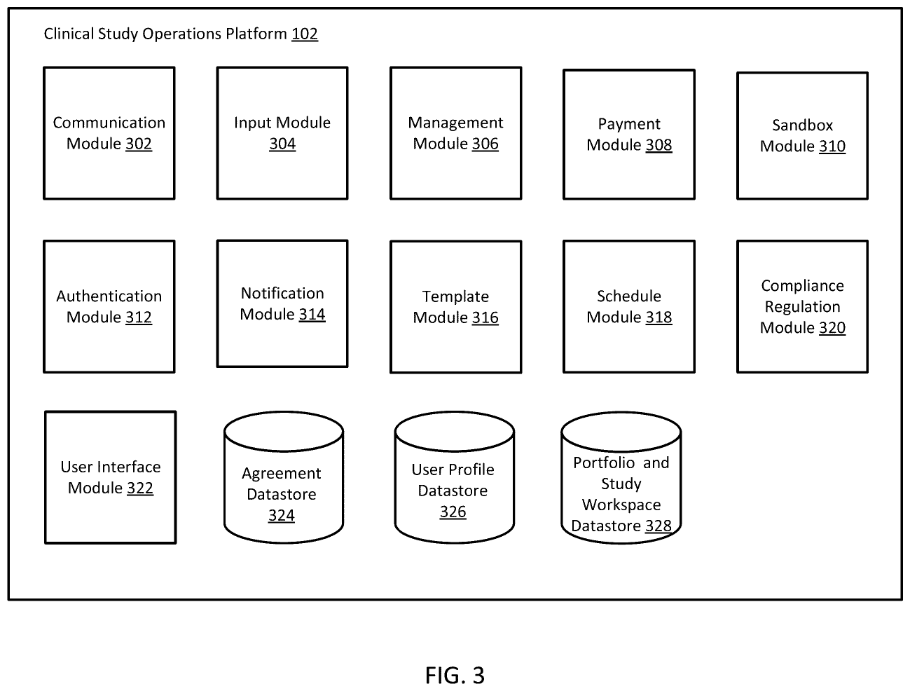

FIG. 3 depicts a block diagram of the clinical study operations platform 102 according to some embodiments. The clinical study operations platform 102 includes a communication module 302, an input module 304, a management module 306, a payment module 308, a sandbox module 310, an authentication module 312, a notification module 314, a template module 316, a schedule module 318, a compliance regulation module 320, a user interface module 322, an agreement datastore 324, a user profile datastore 326, and a portfolio and study workspace datastore 328.

The communication module 302 may send and receive requests or data between any of the modules or data storages of the clinical study operations platform 102. The communication module 302 may send and receive requests or data between any of the clinical study operations platform 102, the first entity system 104, and the second entity system 114.

In various embodiments, the communication module 302 also confirms security by authenticating credentials for an active account, ensures that client data is partitioned such that data can only be accessed by an authorized client only, and the like. In various embodiments, the communication module 302 may enforce encryption of data both at rest (e.g., datastores and databases may be encrypted) and in transit.

The input module 304 may receive requests or data from any of the modules or data storages of the clinical study operations platform 102. The input module 304 may receive requests or data from any of the clinical study operations platform 102, the first entity system 104, and/or the second entity system 114.

In some embodiments, the input module 304 receives information requests from users to receive subscription options, legal agreements, and/or the like. In various embodiments, the input module 304 may receive configuration information (e.g., reference model selections), data, workflow configuration information and the like to configure a platform for a clinical study. In some embodiments, the clinical study operations platform 102 does not receive or create customized source code for a particular entity or clinical study.

The management module 306 may manage various aspects of the clinical study operations platform 102 including receiving request to access a web page associated with clinical study operations platform. In various embodiments, the management module 306 enables configuration of the platform for a particular clinical study. As discussed herein, an authorized user associated with an entity having purchased a subscription may define or configure their platform to support their particular clinical study.

In various embodiments, the management module 306 enables an authorized user to invite other users and/or define roles for the user. Each role may be associated with rights (e.g., to view their and other user accounts, edit user details, data access, ability to receive alerts, which configurations, if any, they are allowed to input to the clinical study operations platform 102, deactivate a user, and the like).

In some embodiments, the user may select a reference model from a list of reference models. For example, the user may review a configuration interface provided by the clinical study operations platform 102. The clinical study operations platform 102 may provide a list of pre-configured reference models to configure the workspace according to the reference model discussed herein. Upon selection of a reference model, the management module 306 may retrieve a pre-determined list of configurations associated with the reference model configuration (e.g., from a datastore), and automatically configure the authorized user's platform according to the list of configurations. In some examples, the selected reference model may be associated with a list of pre-defined configurations that define categories and subcategories for documents, outline metadata fields for indexing and searching, specify document relationships, workflow processes, and compliance requirements enforce by the pre-configured code to ensure compliance with applicable legal and regulatory requirements.

It may be appreciated that an authorized user may also utilize the management module 306 to alter or configure the platform to create new configurations (e.g., as listed above) without providing or creating source code (e.g., in this example, configurations and functions are pre-defined). For example, an authorized user and/or a reference model may further select and provide information from configuration fields, picklists, and trackers.

In some embodiments, the payment module 308 determines the cost of a subscription based on information provided by a user or provides subscription options. In some embodiments, the payment module 308 provides payment options (e.g., invoice payments, payment by others, fractional payments across payment modes, and/or the like), receives payment, confirms payment, validates identity, and/or the like.

In various embodiments, the sandbox module 310 may receive a request from the management module 306 to generate a sandbox study. In some embodiments, authenticated users and one or more other invited users invited by the authorized user may access a sandbox study of artificial data. The sandbox study allows a user to view the clinical study operations platform 102 and test out the features of the clinical study operations platform 102 without impacting live study data. The sandbox study may be used for training.

All templates may be available to test in an optional sandbox study. Templates can be tested by adding a calendar site visit of the associated visit type to the sandbox study, and then using the Site Visit Tracking view to create an eVisit Report for that template. If multiple templates are available for the same visit type then the user may have the choice to choose which template to use upon creation of the report. Note that the sandbox uses fictitious team members, so the sandbox is ideal for testing of templates, but does not enforce the same access control that you find in live studies which have real users assigned.

The authentication module 312 may authenticate a user by comparing the user's login and password with a database of registered users. It may be appreciated that users may be authenticated in many ways including, but not limited to, device identifiers, biometrics, encryption keys, cookies, and/or the like.

In some embodiments, a user may utilize the same or similar credentials (e.g., a username) to access any number of clinical study workspaces for different clients. In one example, a user may be associated with a sponsor or CRO that are sponsoring or managing multiple clinical studies for different entities. The user may access the workspaces they need to manage, supervise, assess, and/or learn about the different clinical studies (e.g., via a clinical study platform) through a single authentication through the clinical study operations platform.

In various embodiments, the template module 316 may generate a portfolio workspace based on information received from the authorized user during the onboarding process. For example, the authorized user may input the name of the company the authorized user works for, as well as subscription add-ons that the authorized user chose in order to generate a portfolio workspace.

In one example, the template module 316 may be configured to import bulk records into the clinical study operations platform 102. The template module 316 ensures that the data being imported is in the correct format to be accepted into the clinical study operations platform 102.

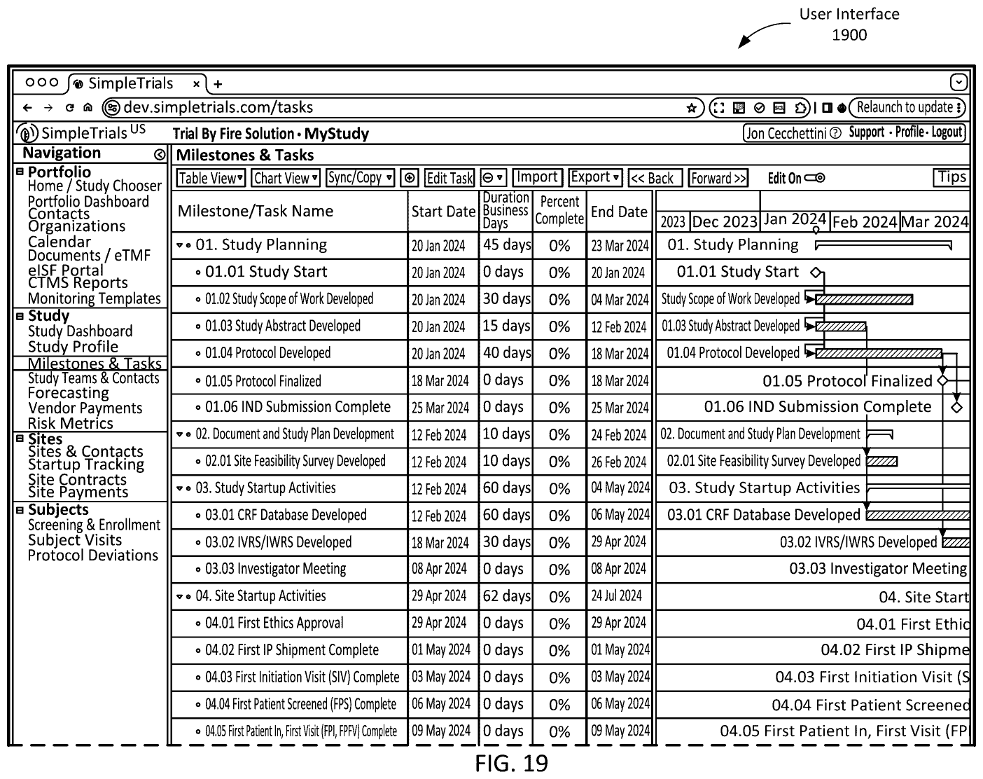

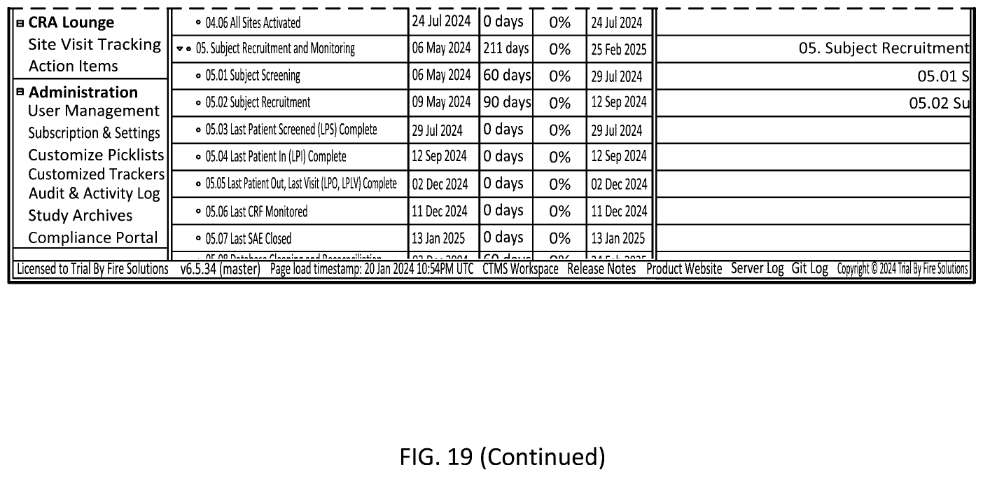

The schedule module 318 may utilize past duration of one or more tasks associated with the clinical trial and a projected start date to provide an estimate of the projected end date. An example of this can be found in user interface 1900 of FIG. 19. The user interface 1900 provides tracking major study-related milestones and tasks. Tasks with a duration of 1 or more days are indicated by horizontal bars showing progress over time. It may be appreciated that an authorized user may define any number of milestones including start dates, duration days, anticipated end dates, and notes. In this example, milestones have a duration of zero days and are indicated by a shape that represents the date a milestone may be or has been achieved. Milestones and tasks are a two-level project plan, with child subtasks representing individual tasks and milestones line items, and parent tasks represent an aggregate of their child subtasks.

In various embodiments, the schedule module 318 may create one or more milestones. These milestones may be internal. Examples of milestones may include milestone to create a clinical study workspace, start a clinical study, a startdate of the milestone (e.g., startdate of the clinical study) and/or the like. The schedule module 318 and/or the notification module 314 may track a target based on the milestone (e.g., based on a date of a milestone). In some embodiments, the schedule module 318 and/or the notification module 314 may provide alerts for upcoming milestones, missed milestones, and/or the like.

In various embodiments, the authentication module 312 may configure rights of one or more users to receive all, some, or specific alerts related to milestones. For example, a user may be given rights to receive notifications or alerts related to a specific milestones of a particular clinical study, rights to receive notifications or alerts related to a particular class of milestones of a particular clinical study, rights to receive notifications or alerts related to a particular milestone or a class of milestones across any number of clinical studies, and/or the like.

Notifications or alerts may include a link or invitation to view the clinical study workspace (and/or a portfolio workspace) to review the missed or upcoming milestone(s), create new milestones (e.g., to make corrections as needed), and/or make configuration changes to future notifications and/or alerts.

The optional compliance regulation module 320 may identify a country that one or clinical trials are taking place in and determine the compliance regulations that need to be followed to maintain the compliance of a clinical study. The compliance regulation module 320 may provide the clinical study operations platform 102 with proper user authentication and access controls, verifying the identity of users trying to access the clinical study operations platform 102 and only allowing valid user with proper credentials can access the system and have access to data they are authorized to view or change. For example, in order to comply with 21 CFR Part 11, the clinical study operations platform 102 may require two-factor authentication or biometric authentication in order to access the CTMS.

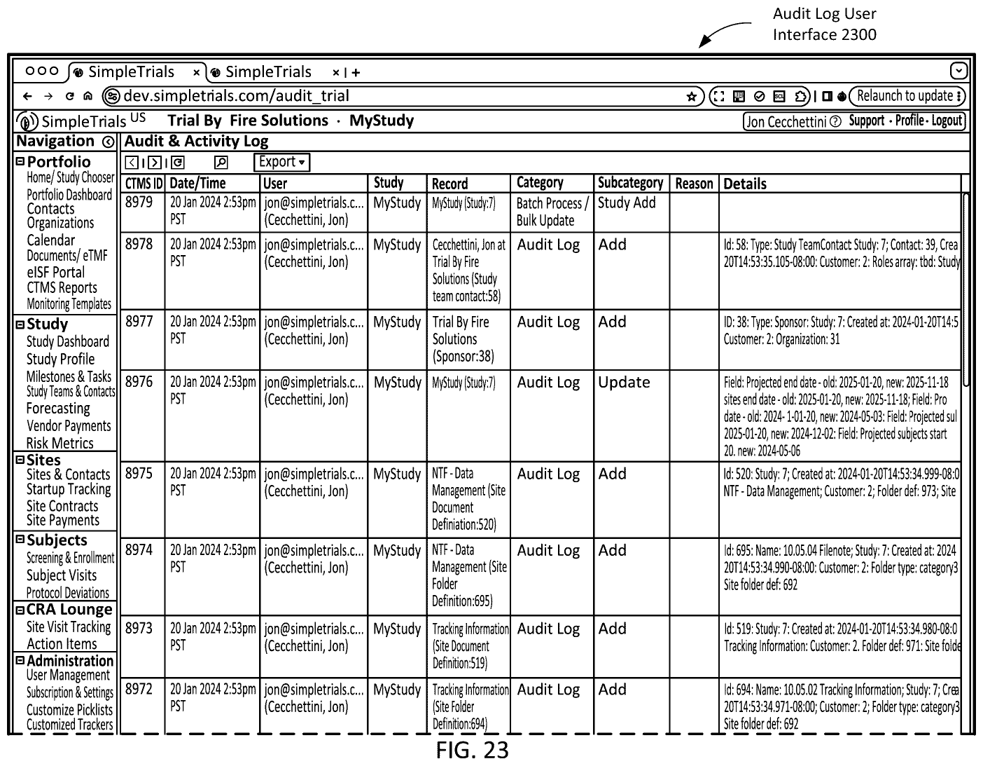

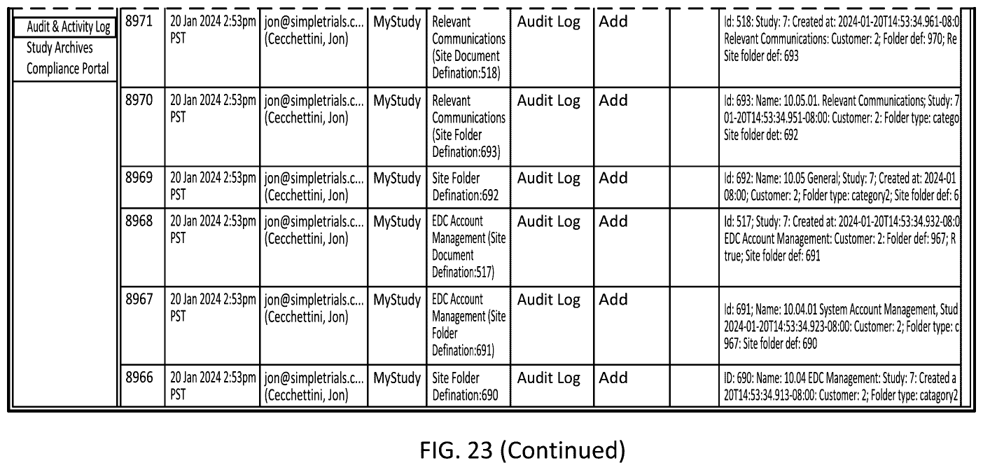

Furthermore, the compliance regulation module 320 may provide that the CTMS clear and concise audit trials to track changes in electronic records and secure record retention for FDA review. The compliance regulation module 320 may provide to the user interface module 322 an audit log similar to that of an example audit log 2300 of FIG. 23 which provides a table of all the changes across a particular clinical trial or a particular portfolio that includes multiple clinical trials taking place in one or more countries.

In some embodiments, the compliance regulation module 320 may enforce security and provide information for audits. In some embodiments, the compliance regulation module 320 authenticates user to view or perform audits. The compliance regulation module 320 may capture change history of data records (e.g., what was changed, date of change, time of change, person or entity making the change, computer in which the change was used to make the change, and the like). The compliance regulation module 320 may also track and capture activity history for any number of users including login, account activities, and the like. The compliance regulation module 320 may allow an authorized user to sort, search, and filter tracked information (e.g., logged data changes, additions, deletions, users who made changes, when changes were made, and the like). In some embodiments, the compliance regulation module 320 will not allow audit trail records to be altered or modified.

A Trial Master File (TMF) is a collection of documents that are used for the management, conduct, and oversight of a clinical trial. The TMF provides a central source of documentation required to demonstrate compliance with regulatory requirements. TMF includes documentation on trial set-up, such as study protocol and amendments and regulatory authority approvals, trial management, such as site training records, informed consent forms from participants from the clinical site, and trial close-out which includes financial and contractual documentation and a final study report which includes a summary of the findings from the clinical site for the clinical trial. The convenience and popularity of the electronic TMF have benefits such as version control, accessibility, and an ease of collaboration between users in different physical geographical locations.

For eTMFs used in the United States, documenting clinical trials being performed in the United States need to obtain and maintain compliance with 21 CFR Part 11 to ensure secure, auditable, and traceable digital records. For eTMFs in the European Union, the EU Clinical Trials Regulation (CTR) accentuates the importance of TMF documentation in clinical trials and clinical sites conducted in the European Union.

The user interface module 322 may provide one or more example user interfaces for the onboarding method of the clinical study operations platform 102. Onboarding may include one or more of account setup, platform orientation, integration support, user configuration of the system/platform, user invitations and role assignments, performance validation, and/or the like. The example user interfaces provided by the user interface module 322 may be sent to an output device of the digital device.

The agreement datastore 324 may be any structure and/or structures suitable for storing data entries or records (e.g., an active database, a relational database, a self-referential database, a table, a matrix, an array, a flat file, a documented-oriented storage system, a non-relational No-SQL system, an FTS-management system such as Lucene/Solr, and the like). The agreement datastore 324 may receive a request from an authorized user or a potentially subscribing user to access legal agreements stored in contract entries. Each contract entry may represent a legal agreement that the clinical study operations platform may require one or more users associated with various aspects of the clinical trial process to consent or otherwise agree in order to utilize the clinical study operations platform 102. In some embodiments, the agreement datastore 324 includes a subscription agreement between the clinical study operations platform 102 and the user authorizing the subscription. In one example, the agreement datastore 324 includes a log of consent to any number of legal agreements (e.g., Terms of Use, Privacy Policy for Subscribers, and a Privacy Policy).

In some embodiments, one or more contract entries may represent a legal agreement that one group of users associated with the clinical trial process requires another group of users associated with a different aspect of the clinical trial process to consent or otherwise agree. For example, a company that is developing a particular drug may require a clinical research coordinator and other members of the team at a clinical site to sign a legal agreement (for example, to effectively sign one or more legal documents) before proceeding with the clinical trial for the particular drug.

The user profile datastore 326 may be any structure and/or structures suitable for storing data entries or records (e.g., an active database, a relational database, a self-referential database, a table, a matrix, an array, a flat file, a documented-oriented storage system, a non-relational No-SQL system, an FTS-management system such as Lucene/Solr, and the like). In some embodiments, the user profile datastore 326 may receive a request from the management module 306 to create a user profile entry. Each user profile entry may include information such as a user's login and password. The login and password may be associated with the user's account. In some embodiments, the user profile entry includes data such as account subscription setting one or more clinical trials associated with the user as well as a user type which determines read/write privileges of the user for one or more of the clinical site, clinical trial, or portfolio or some combination therein.

A user interface may allow a user to configure an account with the clinical study operations platform 102. The user may select a role for themselves and/or other users (e.g., to be invited) from a list (e.g., pull-down menu, radio button menu, or the like) of different user types (e.g., a pull-down menu of admin, manager, associate, and viewer).

User types may be categorized by, but not limited to, admin, manager, associate, and viewer. An administrator or admin may have access to all data across all clinical trial studies and portfolios, access compliance data and reports, generate study archives, and audit and activity logs. A manager may have read/write access to study definitions, such as parameters or properties of the participants pertinent to the clinical trial or study. While an associate may have read/write access to clinical data at a particular clinical trial site. A viewer user type only has access to view clinical data from the clinical study operations platform 102. In some embodiments, the viewer may be restricted to a clinical site or a particular clinical trial that includes multiple clinical sites.

The portfolio and study workspace datastore 328 may be any structure and/or structures suitable for storing data entries or records (e.g., an active database, a relational database, a self-referential database, a table, a matrix, an array, a flat file, a documented-oriented storage system, a non-relational No-SQL system, an FTS-management system such as Lucene/Solr, and the like). The portfolio and study workspace datastore 328 may receive a request from the management module 306 to create or update a portfolio workspace entry and/or a study workspace entry. Each study workspace entry may be associated with one clinical trial. The study workspace entry may include data associated with a particular clinical trial as well as data associated with one or more clinical trial sites associated with the particular clinical trial. Multiple clinical sites may be associated with each clinical trial. One clinical trial may be associated with multiple clinical trials. Data stored in study workspace entries may include data such as a projected start date of the study and study identification data such as the name of the study and the sponsor. Other data stored in the study workspace entries may include documents associated with the study and may include the eTMF.

In some embodiments, the portfolio and study workspace datastore 328 may receive a request from the management module 306 to create or update contact entries. Each contact may include identification and contact information to get in touch with different members of the clinical trial process.

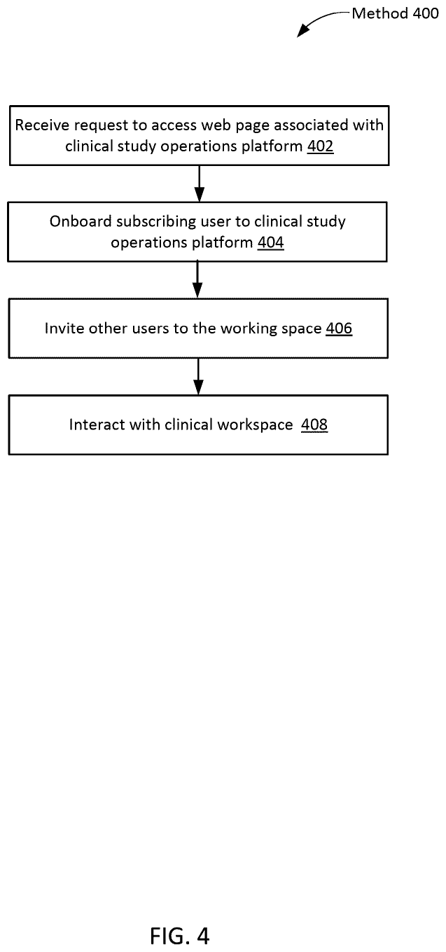

FIG. 4 depicts a flowchart of a method 400 of onboarding and navigation of the clinical study operations platform 102 according to some embodiments. In this example, the clinical study operations platform 102 is a multi-tenanted software. Authorized users from different sponsors may share a common access with specific privileges to an instance of the clinical study operations platform 102 according to their respective subscription plan.

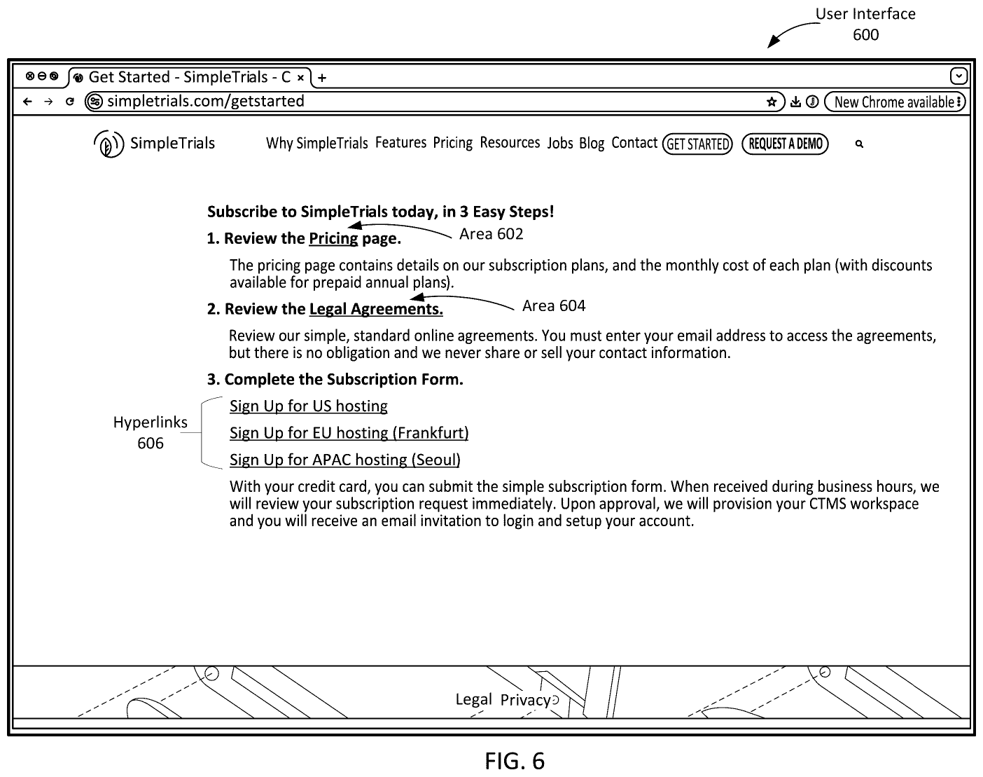

In step 402, a potential user navigates to the clinical study operations platform 102 via a network (e.g., the Internet). The clinical study operations platform 102 may host a user interface 600 of FIG. 6. The user interface 600 may depict a webpage of information associated with subscription and functions of the clinical study operations platform 102. In the example of FIG. 6, a user interface 600 is depicted showing information about a subscription service available on the clinical study operations platform 102. In this example, there is information and links for pricing, legal agreements, and sign up forms. In other examples, the user interface 600 may provide pricing information directly, tutorials, how the system works, downloadable documents (e.g., downloadable legal agreements and/or sign up forms), fillable fields to review and sign legal agreements and sign up information, and/or the like.

In the example of FIG. 6, the potential user may interact with area 602 of the user interface 600 to access a webpage for information on the pricing associated with a subscription to access the multi-tenanted clinical study operations platform 102.

The user may interact with area 604 of the user interface 600 to access a webpage to access legal agreements. The user may interact with one of the multiple hyperlinks 606 of the user interface 600 to start the onboarding process. The legal agreements, pricing, and the like may differ depending on needs, subscription, demographics, type of study, and/or geographic area of the authorized user(s). In some embodiments, the geographic area of the clinical trial sites or the government regulator that the drug trial is attempting to pass a drug determines the subscription form and/or legal agreements that are used. When the user interacts with the multiple hyperlinks 606, the method 400 may proceed to step 404.

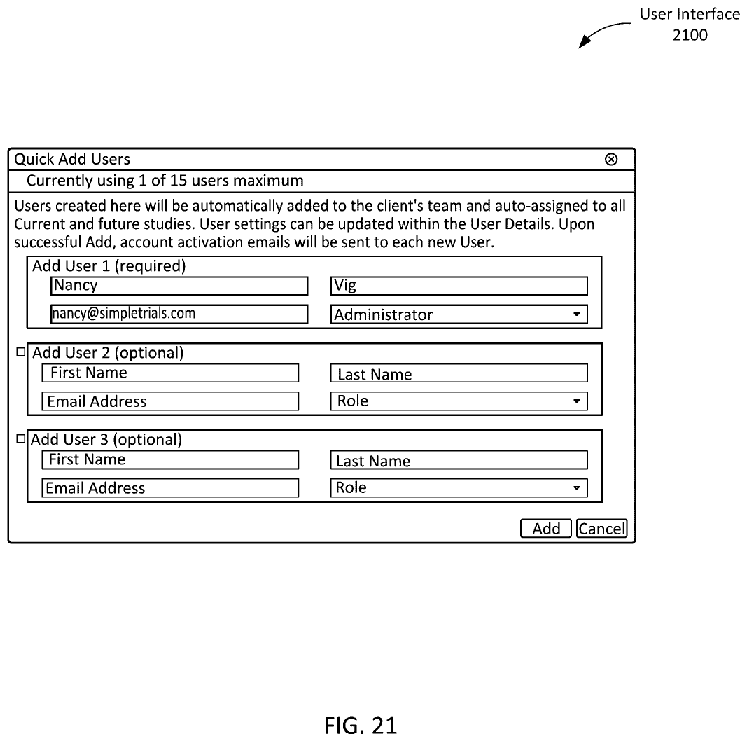

In step 404, the user executes the steps of the method to onboard the authorized user to the clinical study operations platform 102. Examples of the onboarding process are described herein. In step 406, the authorized user invites other users to the portfolio workspace or study workspace. In one example, an authorized user may invite other users to the clinical study operations platform 102 (to engage with a clinical study workspace and/or a portfolio workspace) by interacting with user interface 2100 of FIG. 21. The user interface 2100 includes fields in which an authorized user or associated user may invite others (e.g., by populating the name, email address, and role(s) in the particular study workspace or portfolio workspace for the invited user). Invited users or the user inviting others may be or include member(s) of the government regulatory agency.

In some embodiments, there may be a single clinical trial workspace. In this example, the inviting user may invite others to engage or access with the clinical trial workspace.

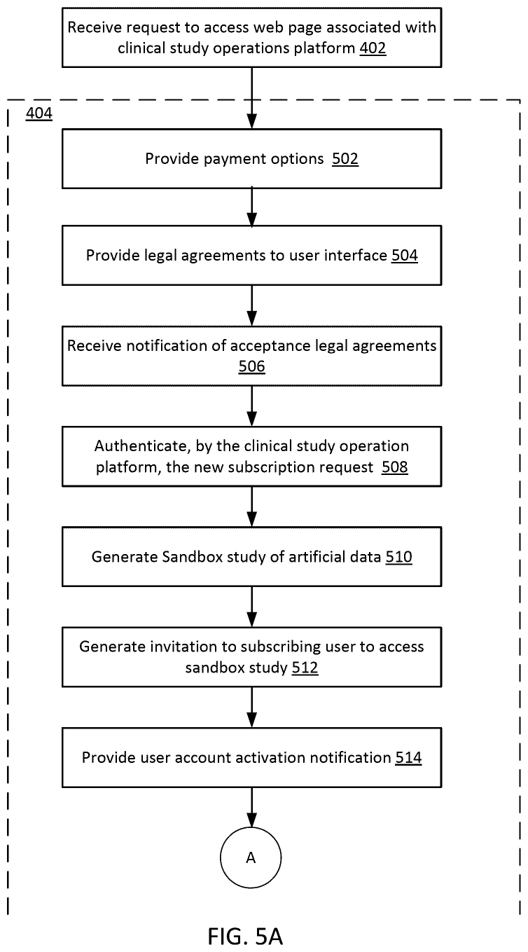

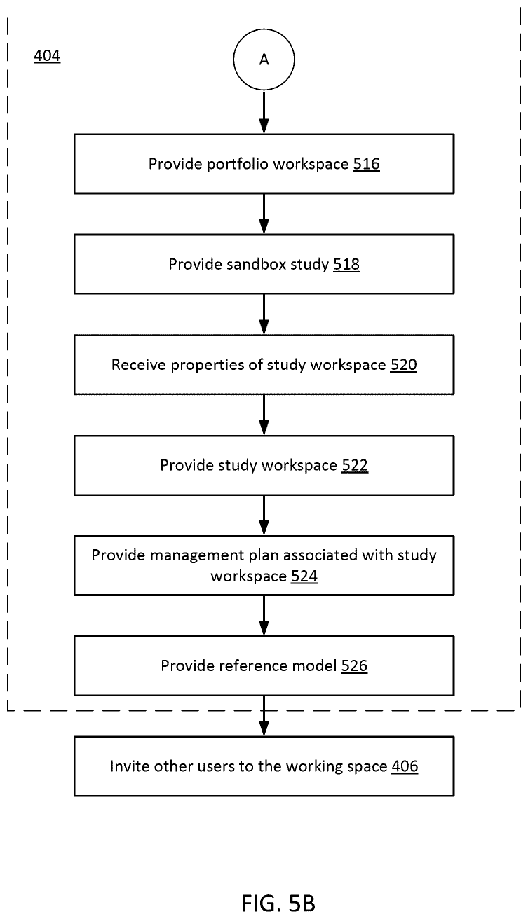

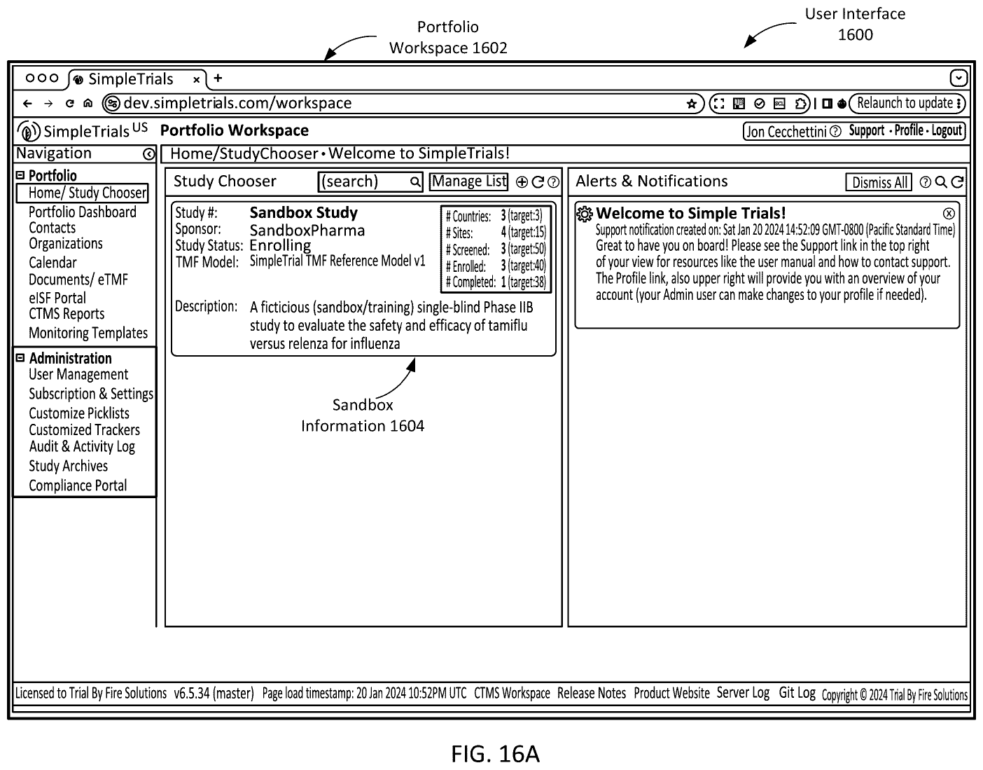

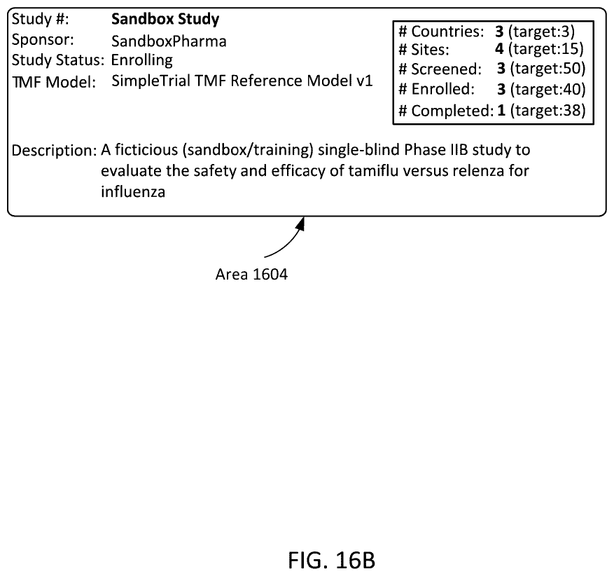

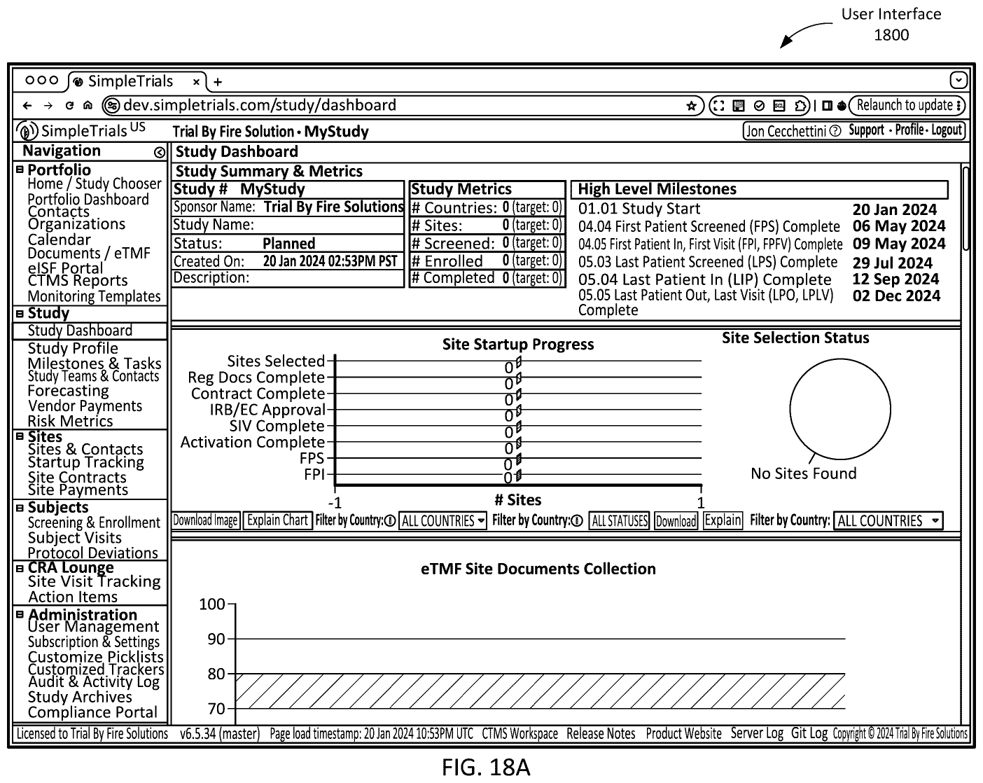

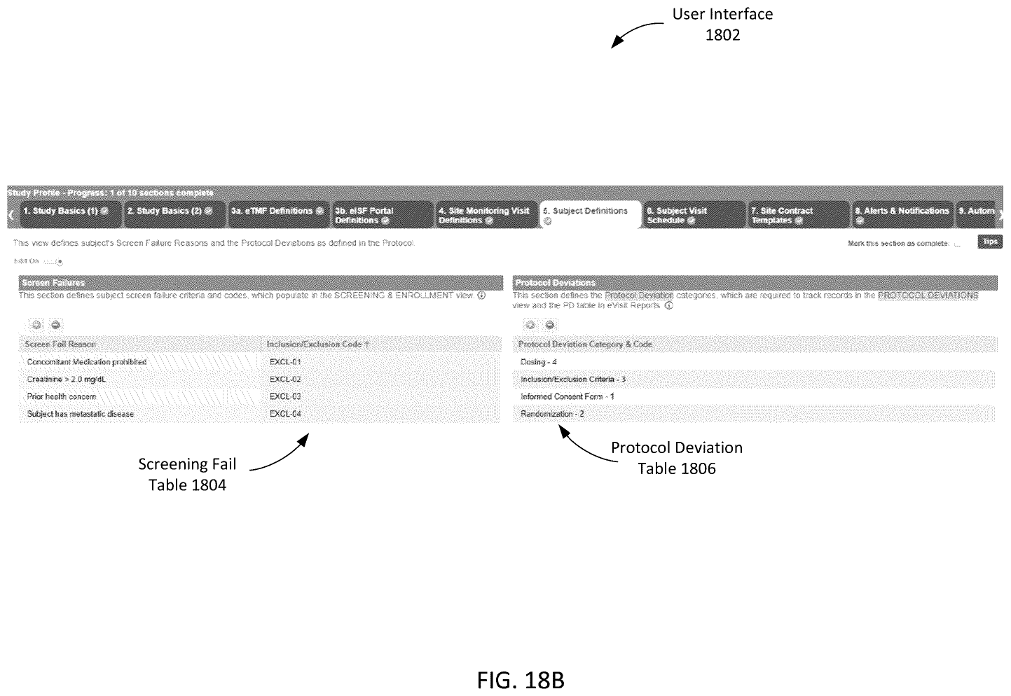

When there are multiple clinical trials for a particular client, a portfolio workspace may display and manage data across multiple clinical trials. In this example, the example portfolio workspace can be seen in user interface 1600 of FIG. 16A. Data from one or more clinical trials becomes a portfolio of cross-study data. A study workspace provides a summary for management of data of a particular clinical trial. Data such as milestones and site startup progress may be created and statuses provided. An example study workspace can be seen in a user interface 1800 of FIG. 18A. Further details of step 404 will be discussed with regard to FIGS. 5A and 5B. The inviting user may invite another user to engage with one or more clinical trials and/or the portfolio workspace.

In step 408, the subscribing user or other authorized users may interact with the clinical study operations platform 102 to plan, manage, view, and/or report on clinical trials (depending on the needs and rights granted to the authorized user). The clinical study operations platform 102 may provide participant visit calendaring, screening and enrollment, and/or provide training videos for clinical staff and/or clinical participants. In some embodiments, the other user may be site staff, clinical research coordinator, sponsor, or contract research organization. In various embodiments, the other users may be regulatory authorities working for the EMA or FDA, monitoring safety data and ensuring that clinical participants' rights are protected. Additionally or alternatively, these users working for regulatory authorities may review and/or approve study protocols.

A sponsor may provide oversight. A sponsor may assign specific rights to particular users for oversight. In some embodiments, sponsor oversight may be an attribute of the clinical study operations platform 102 designs for authorized users using a CRO to run and manage clinical trials or studies. Sponsor oversight may, in some embodiments, allow users to determine which clinical trials or studies to track and changing the fields to track.

In various embodiments, a request may be made by authorized users who are authorities working for the EMA or FDA. Periodically, these users require access to compliance, quality and validation documentation from the clinical study operations platform 102. These users may require access to clinical studies across different sponsors, portfolio workspaces, and study workspaces. The user may send a request to the clinical study operations platform 102 for access to a compliance portal of the navigational panel of the clinical study operations platform 102.

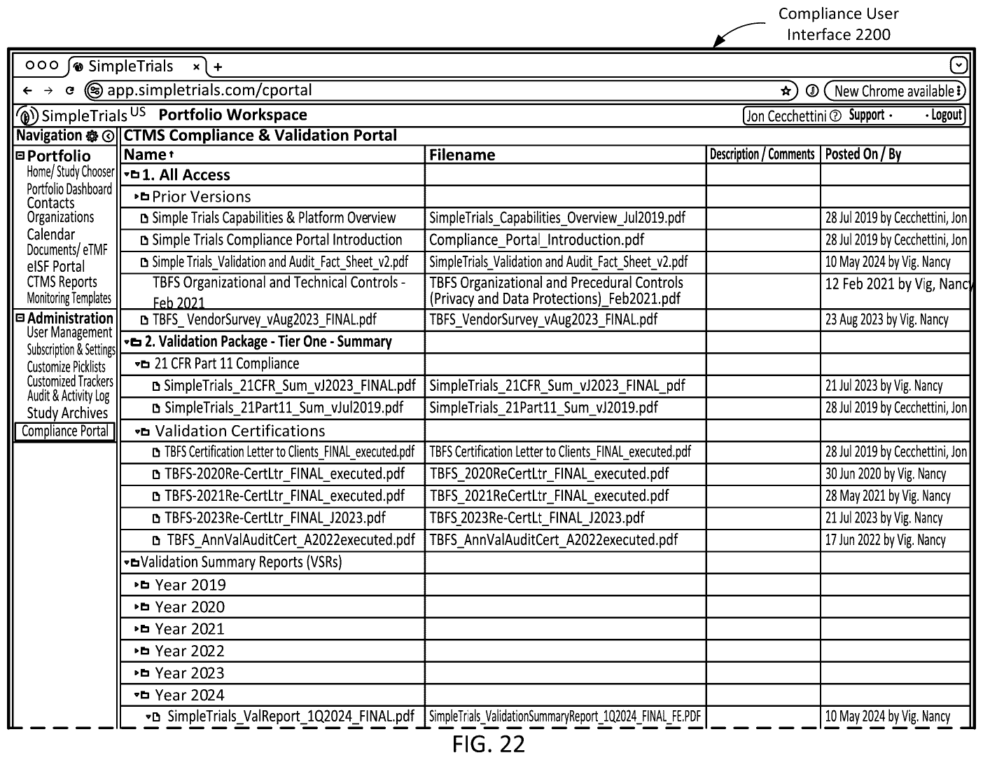

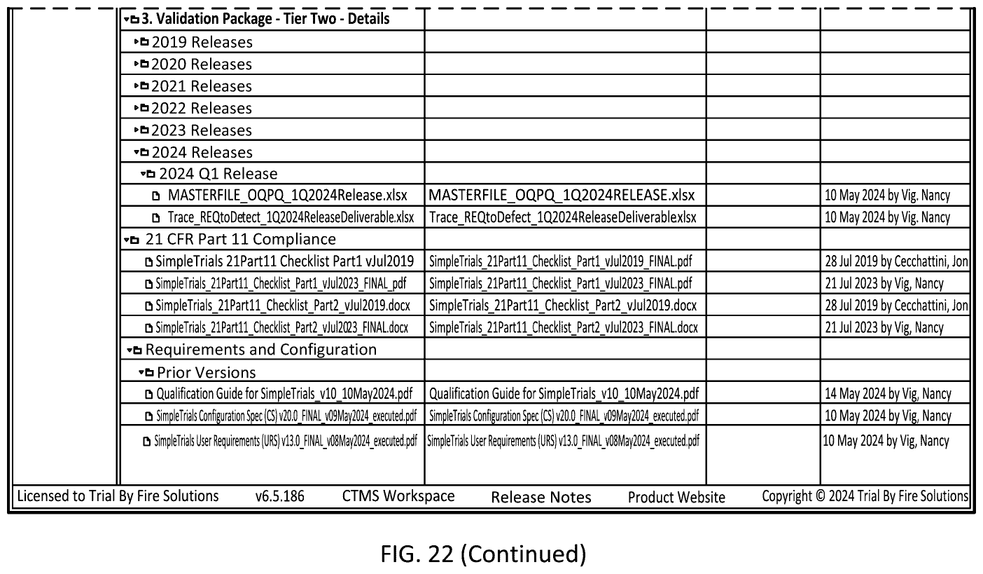

An example of the compliance portal user interface can be seen in compliance user interface 2200 of FIG. 22. The compliance regulation module 320 may receive the request from an authorized user and send a request to the portfolio and study workspace datastore 328 for study workspace entries that fulfill the requirements of one or more compliance regulation(s). Compliance user interface 2200 of FIG. 22 may include a table with rows listing documents which are compliant to different government regulations such as 21 CFR Part 11 Compliant documents.

In another example, step 408 includes a request made by users who are authorities working for the EMA or FDA. As a part of the regulatory compliance, the members of the clinical trial may be required to provide an audit log or report to track all data record changes in the system over a particular period of time. This information may be utilized to maintain and track the integrity of the clinical data. In this example, the user may send a request to the management module 306 for an audit log. The management module 306 may receive this request and send it to the schedule module 318. The schedule module 318 may be configured to identify data records that were updated or created over a particular period of time and send a request to the portfolio and study workspace datastore 328 for study workspace entry that correspond to the identified data records. The identified data records may be provided to the user interface module 322 to be arranged in an audit log. An example of which can be seen in the audit log 2300 of FIG. 23.

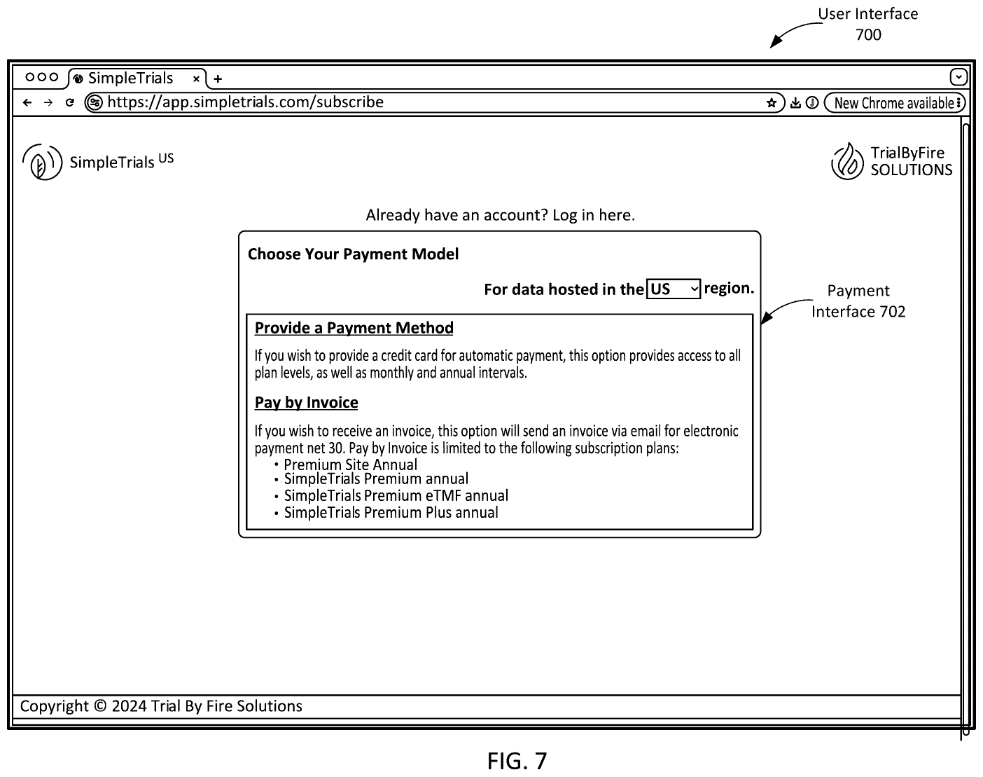

FIGS. 5A and 5B depict a flowchart for subscribing, onboarding, and configuration of the clinical study operations platform for a particular clinical study according to some embodiments. After a user has reviewed subscription terms and/or legal agreements, in step 502 the management module 306 may provide payment options to the subscribing user (or authorized user) via the user interface. In one example, the clinical study operations platform 102 may provide user interface 700 of FIG. 7. A payment interface 702 of FIG. 7 may provide the subscribing user with choices of different payment methods (e.g., in FIG. 7 by credit card or invoice). It may be appreciated that there may be any number of different payment models (e.g., by scale or size of the clinical trial, number of users, number of invites, number of people interacting with the platform, processing required, storage required, and/or the like). Further, it may be appreciated that different payment models may be utilized depending on usage (e.g., the usage of the clinical study operations platform 102 for this subscription is greater than initially expected or used earlier in the study).

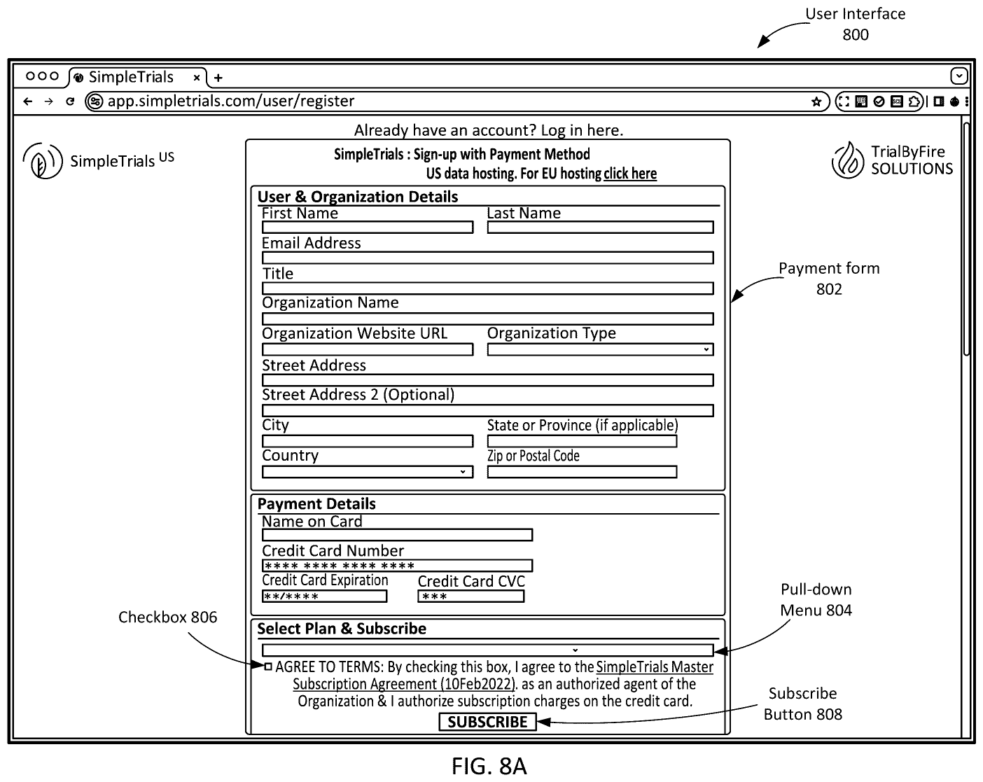

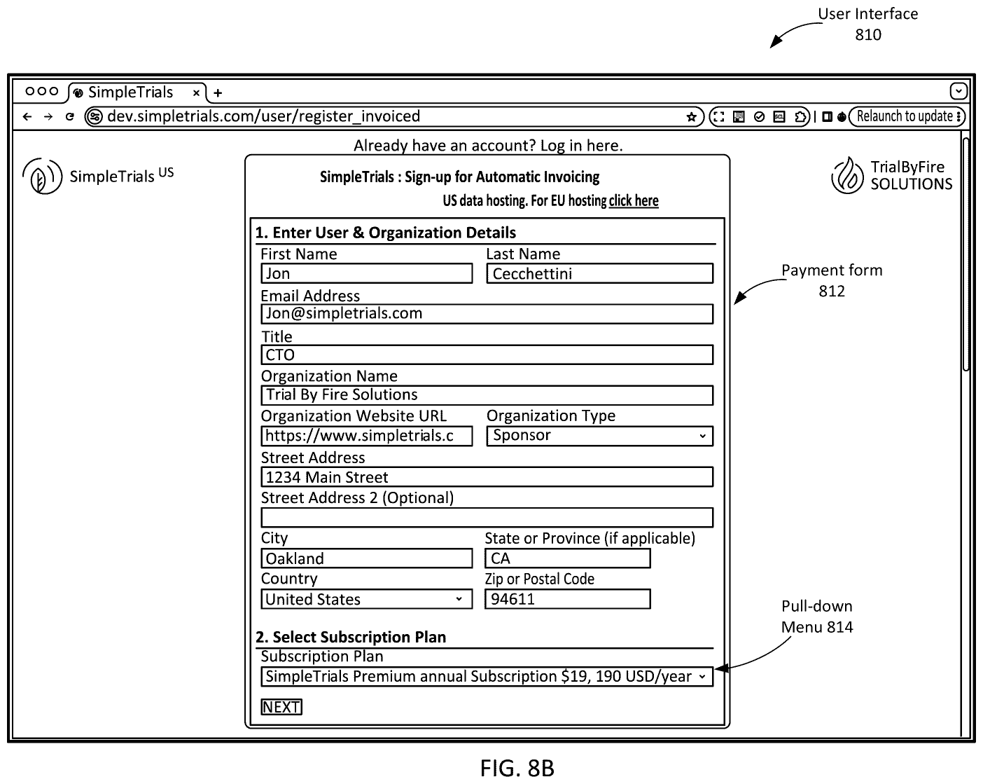

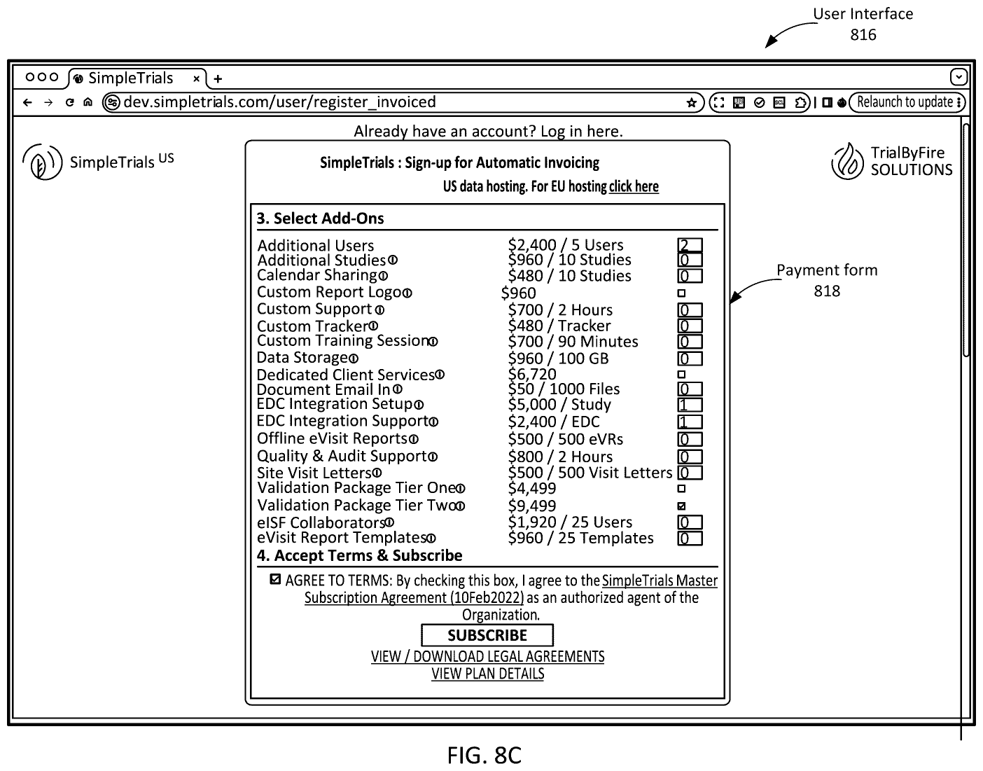

If the subscribing user chooses to pay by credit card, the clinical study operations platform 102 may provide a user interface 800 of FIG. 8A. The subscribing user may provide information in the fields of the payment form 802. For example, the payment form 802 may require identification information from the subscribing user, information such as full name, organization name, and address, as well as credit card information.

In addition to inputting identification information, the user may choose the subscription plan and the number of users on the plan by interacting with a pull-down menu 804 and provide an electronic consent of a subscription agreement by interacting with checkbox 806. After completing these tasks, the subscribing user may interact with a subscribe button 808.

In response to receiving the identification information from the subscribing user, the management module 306 may send a request to the user profile datastore 326 to create a user profile entry.

If the subscribing user chooses to pay by invoice, the user interface module 322 may provide user interface 810 of FIG. 8B. Similar to the user interface 800 of FIG. 8A, the user interface 810, includes a payment form 812. The payment form 812 may require identification information from the subscribing user, information such as full name, organization name and address as well as credit card information. In addition to inputting identification information, the user may choose the subscription plan and the number of users on the plan (e.g., by interacting with a pull-down menu 814).

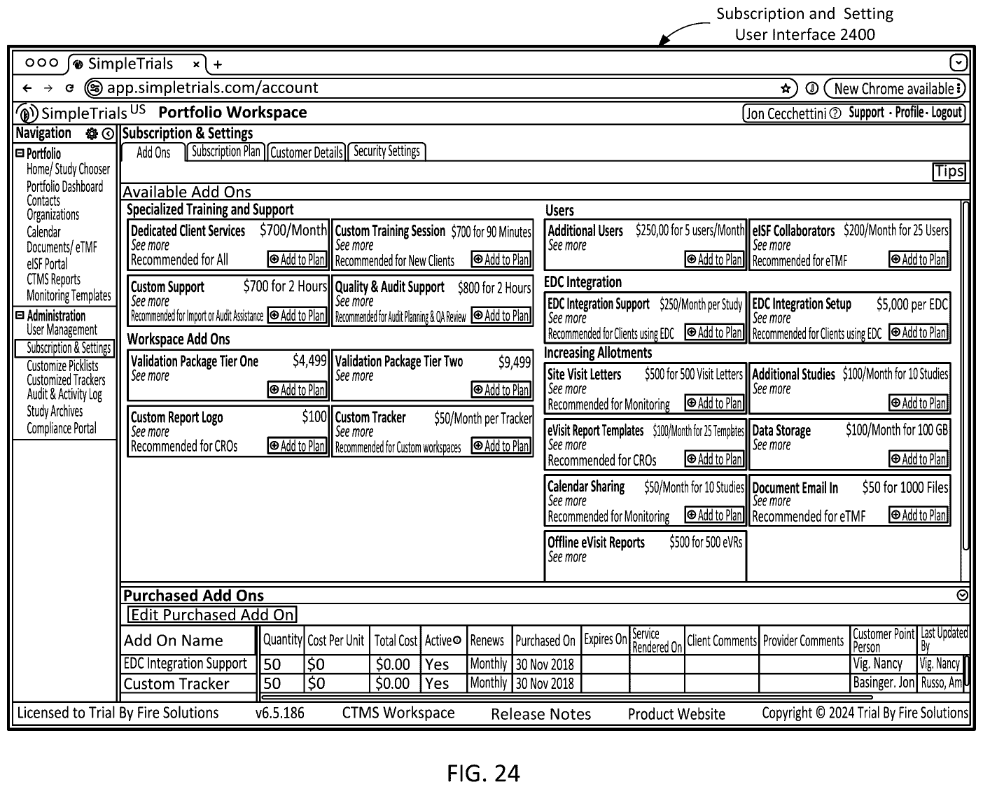

Example user interface 816 of FIG. 8C includes payment form 818 which includes subscription add-ons including options to add additional users options, calendar sharing, and site visit letters and the like. In some embodiments, subscription add-ons may be added or removed on demand. The user may navigate to the Subscription & Settings in the navigational panel and be given access to a webpage associated with the clinical study operations platform 102 that allows users to access their current subscription to make any changes. An example of this webpage can be seen in a subscription and setting user interface 2400 of FIG. 24.

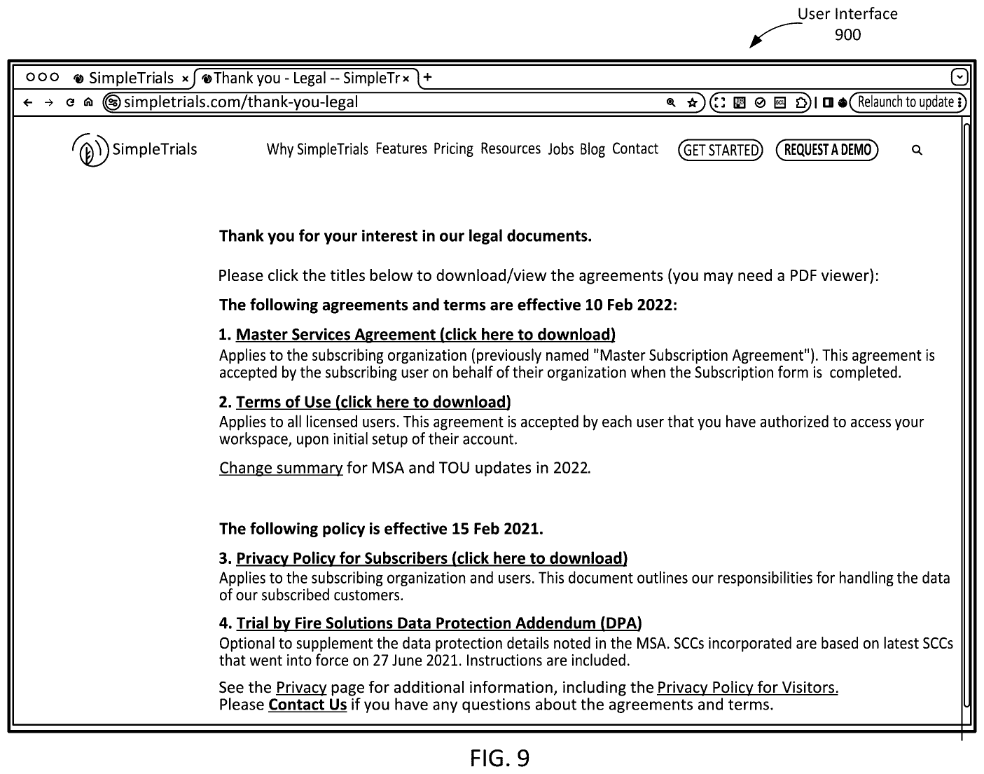

In step 504, the management module 306 may send a request to the agreement datastore 324 to retrieve and provide any number of applicable legal agreement(s). In one example, the management module 306 may provide user interface 900 of FIG. 9 via the user interface module 322. A subscribing user may be required to read and/or accept the terms of the agreement in order to proceed with the onboarding of the subscriber to the clinical study operations platform 102. The agreement datastore 324 may retrieve a particular agreement based on any information. For example, the management module 306 may determine the particular contract agreement to provide to the user interface module 322 based on the location of the server hosting the clinical study operations platform 102. In some embodiments, the location may be determined (e.g., based on the country or countries the clinical trial or drug trial) the required regulation form or agreement.

The user may click an indication to accept, digitally sign, or otherwise execute the legal agreement by providing an electronic signature to a webpage providing access to terms of the legal agreement. In some embodiments, an indication for consent (e.g., click box indicating acceptance), electronic signature, or digital signature may be sufficient. The clinical study operations platform 102 may determine, based on the required legal agreement, subscription, or study type, if different legal execution(s) of the agreement (e.g., manual signature, digital signature, ID requirements, and/or the like) are required. In one example, a manual or handwritten signature is required.

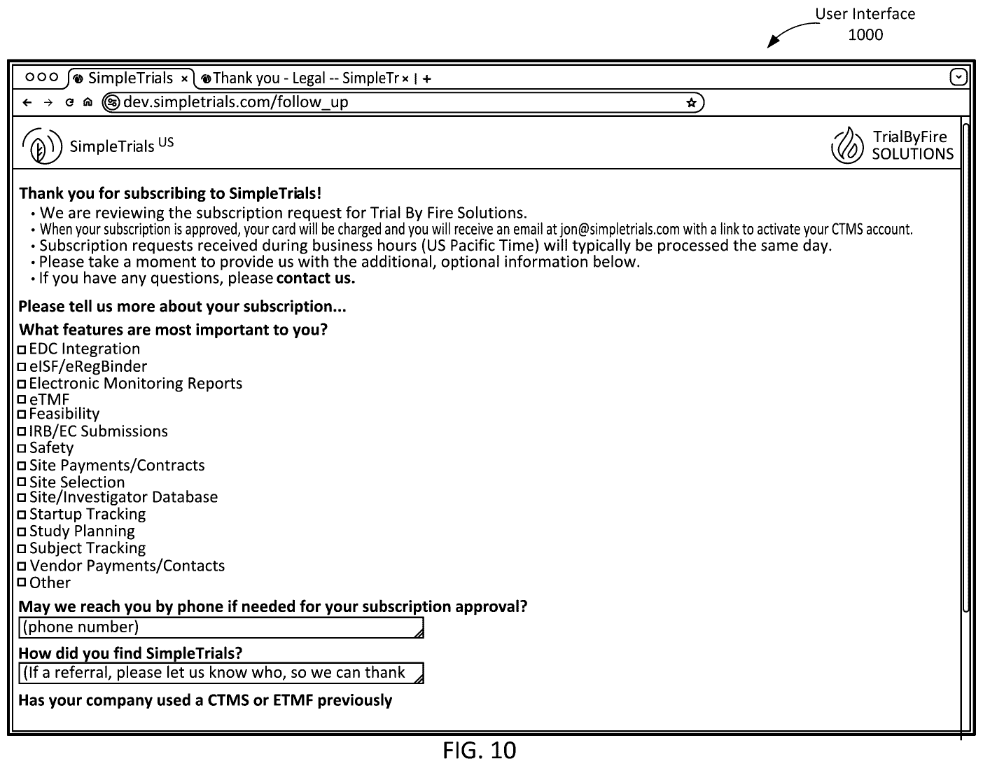

In step 506, the management module 306 may receive notification that the subscribing user agreed to the terms of the legal agreement. In response to receiving the notification, the management module 306 may send a request to the agreement datastore 324 to provide user interface 1000 of FIG. 10 to the user interface module 322. As the user determines the subscription to choose and, optionally, any subscription add-ons, the payment module 308 may calculate the total cost. In some embodiments, the user interface 1000 is provided as a confirmation or receipt of the successful subscription to the clinical study operations platform 102 by the subscribing user.

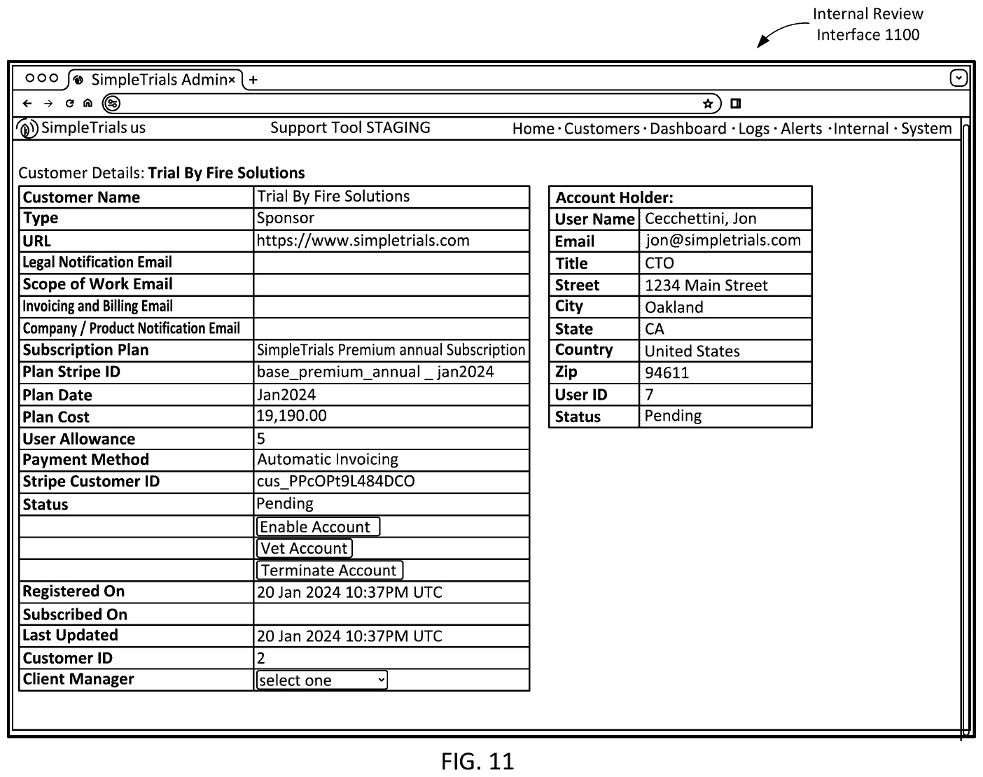

In step 508, an email notification is optionally sent to an employee of an organization associated with the clinical study operations platform 102 (e.g., for internal review before approving a subscription). An example of the email notification can be seen in the internal review interface 1100 of FIG. 11. The employee may receive the email notification and review the subscription and the details provided by the subscribing user, such as company name, name of the subscribing user, etc. Based on the review results, the employee may enable the account to approve the subscription, vet the account (e.g., which may put the subscription on hold while further vetting is performed), or terminate the account, which voids the sign-up. When the subscription has been approved, the method proceeds to step 510.

In some embodiments, the employee may not be associated with or have a stake in any one of the first entity system 104 or the second entity system 114. In this example, the employee may be part of a company that developed the clinical study operations platform 102. In some embodiments, step 508 may be performed by the management module 306 or the authentication module 312 of the clinical study operations platform 102.

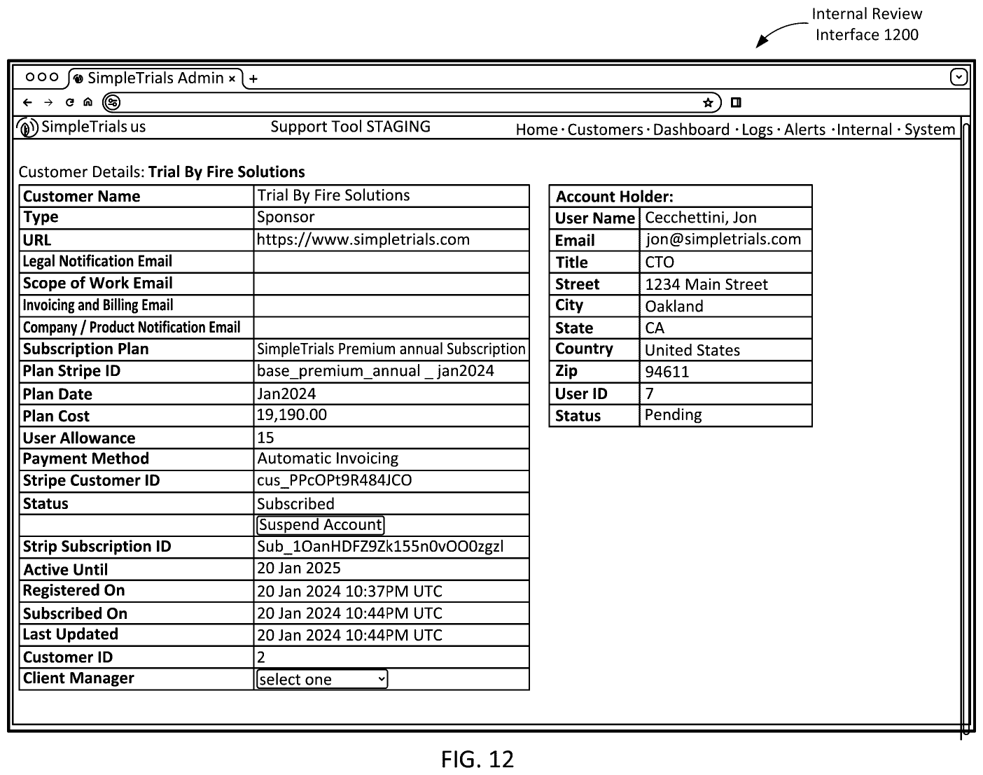

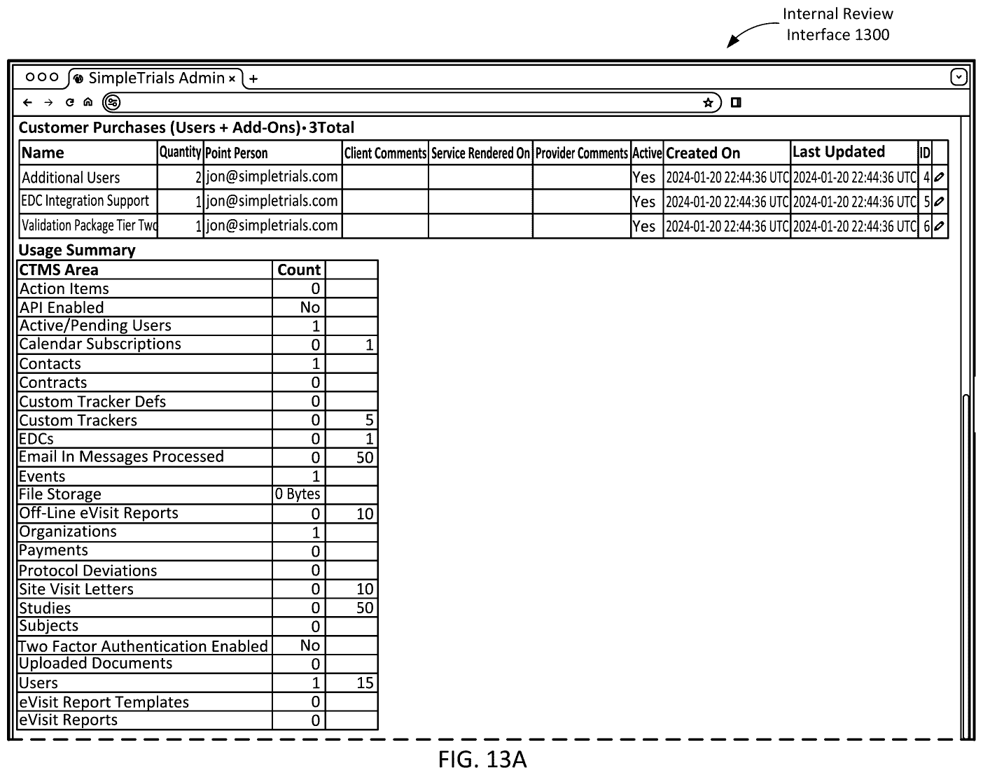

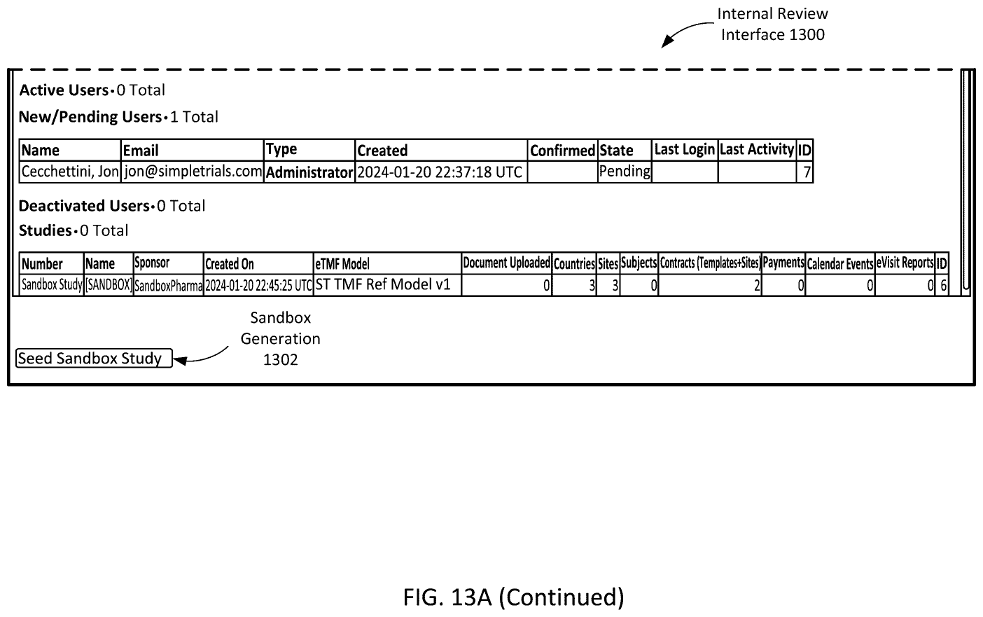

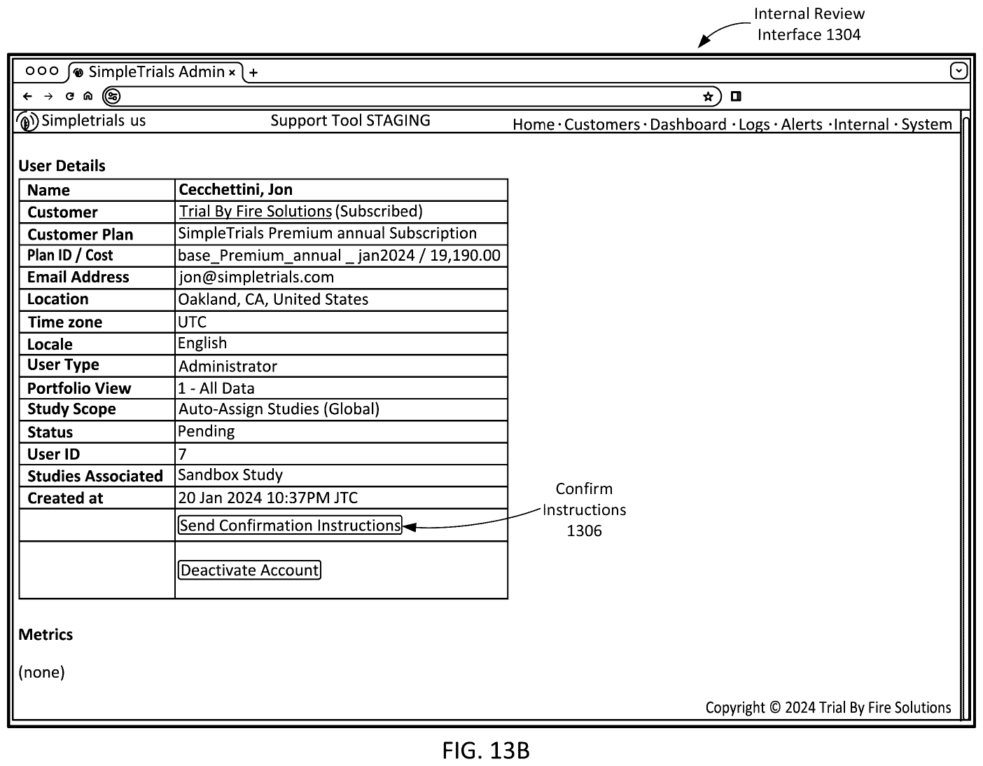

As a part of an internal review of the subscription, an internal review interface 1200 of FIG. 12 may be provided to a supervising employee or user associated with the clinical study operations platform 102, as seen in step 510. An internal review interface 1300 of FIG. 13A may be provided to an authorized supervising employee. The authorized employee may review to verify the subscription add-ons that the subscribing user signed up for.

Upon approval, the supervising employee may provide confirmation instructions to the user regarding approval of the subscription. An example internal review interface 1304 of FIG. 13B is displayed showing the interface that may be interacted with by the supervising employee that approved the subscription.

In step 510, the user may be given the option to create a sandbox study. As discussed herein, the sandbox is an opportunity for the user to experiment and learn the interface. The sandbox study may use artificial data.

In step 512, the management module 306 may receive notification that the account and/or the account information has been vetted or reviewed (e.g., the account or information has been approved).

After receiving this notification, the management module 306 may optionally send a request to the sandbox module 310 to create a sandbox study. In some embodiments, The user may choose to have a sandbox created or may opt to skip the sandbox. If the sandbox is opted, the sandbox module 310 may use a previously configured model for clinical trial management. After generating the sandbox study, the authorized user who can interact with the approved platform associated with the approved subscription may configure and interact with confirmation instructions 1306.

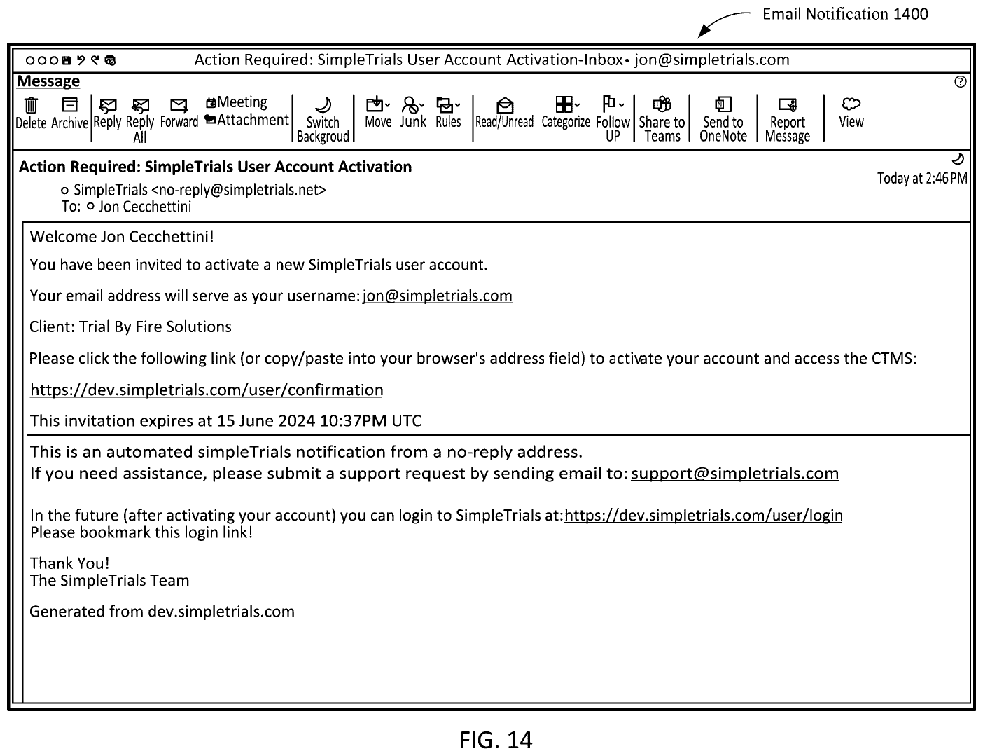

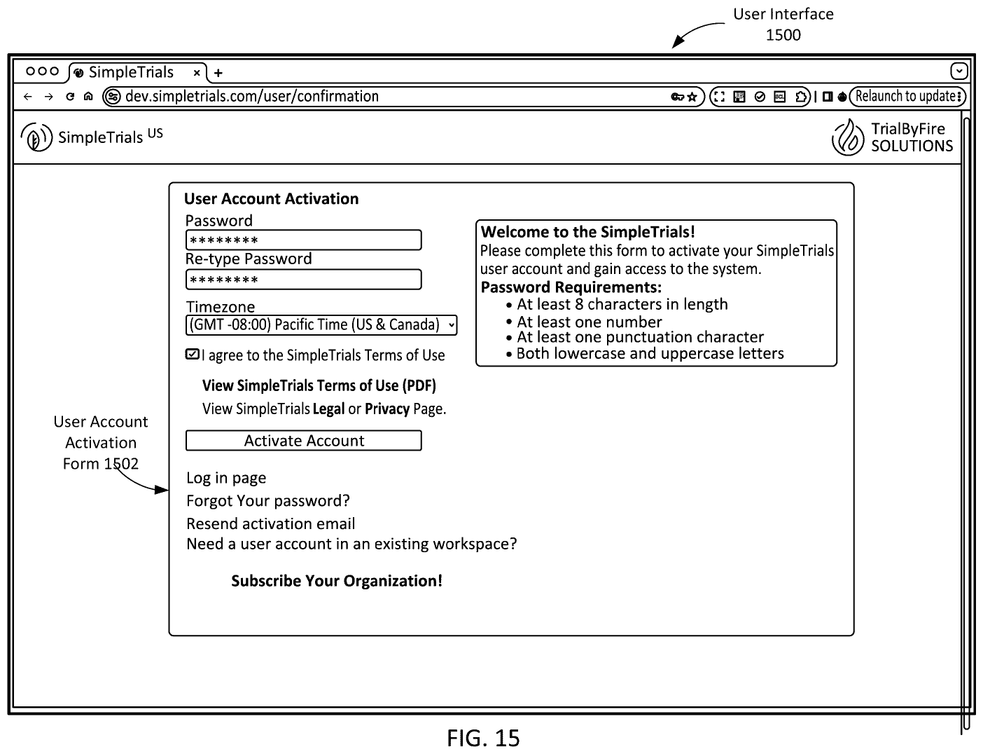

In step 514, the notification module 314 sends an email notification such as email notification 1400 of FIG. 14. The email notification may be sent to an email address of the subscribing user of activation of their account with the clinical study operations platform 102. The user may interact with the hyperlinks in the email notification 1400. In response, the management module 306 may receive a request to access a user account activation web page. An example of the user account activation webpage can be seen in user interface 1500 of FIG. 15. The user interface 1500 includes user account activation form 1502, which includes fields that allow the subscribing user to provide a password. In some embodiments, a user may use other ways to authenticate or secure a user account, such as biometrics, encryption keys, and cookies. In response to the user inputting their password, the management module 306 may send a request to the user profile datastore 326 to update or create a user profile entry associated with the authorized user.

Alternately or additionally, in step 514, the notification module 314 may provide a notification (e.g., email, alert, slack message, or the like) to the backend to indicate that an account is created or needs to be created by the clinical study operations platform 102.

In some embodiments, in step 514, the notification module 314 provides and/or generates an invitation to a subscribing user to access a workspace (e.g., a clinical study workspace and/or a portfolio workspace).

In step 516, the template module 316 may generate a portfolio workspace based on information received from the authorized user during the onboarding process. For example, the authorized user may input the name of the company the authorized user works for, as well as subscription add-ons that the authorized user chose in order to generate a portfolio workspace. It may be appreciated that any functions or steps may occur in any order (e.g., subscription add-ons may be selected at the time of subscription selection, and both may be approved by the supervising employee).

It may be appreciated that in some instances, an authorized user or other user invited to the clinical study operations platform 102 by the authorized user may import data from an external source to the clinical study operations platform 102. For example, a large pharmaceutical company preparing for a large clinical trial may wish to import a large number of contacts such as sponsors, research coordinators, and site staff into the clinical study operations platform 102. The template module 316 may be configured to import bulk records into the clinical study operations platform 102 by providing the bulk data in a data import template.

In some embodiments, each portfolio workspace may include a sandbox study to allow users to view the clinical study operations platform 102 and test out the features of the clinical study operations platform 102 without impacting live study data. An example of a portfolio workspace can be found in the user interface 1600 of FIG. 16A, which includes a portfolio workspace 1602.

In step 518, the sandbox module 310 may send a request to the user interface module 322 to provide the sandbox study to the authorized user. The user may interact with sandbox information 1604 to obtain more information regarding a sandbox study. Referring to FIG. 16B, which shows the sandbox information 1604 in greater detail, provides the name of the study, the sponsor of the clinical study, and the current status of the study. Furthermore, the sandbox information 1604 includes further details regarding the type of trial master file (TMF) model used by the particular clinical study. Furthermore, the sandbox study summary provided in the portfolio workspace 1602 of FIGS. 16A and 16B

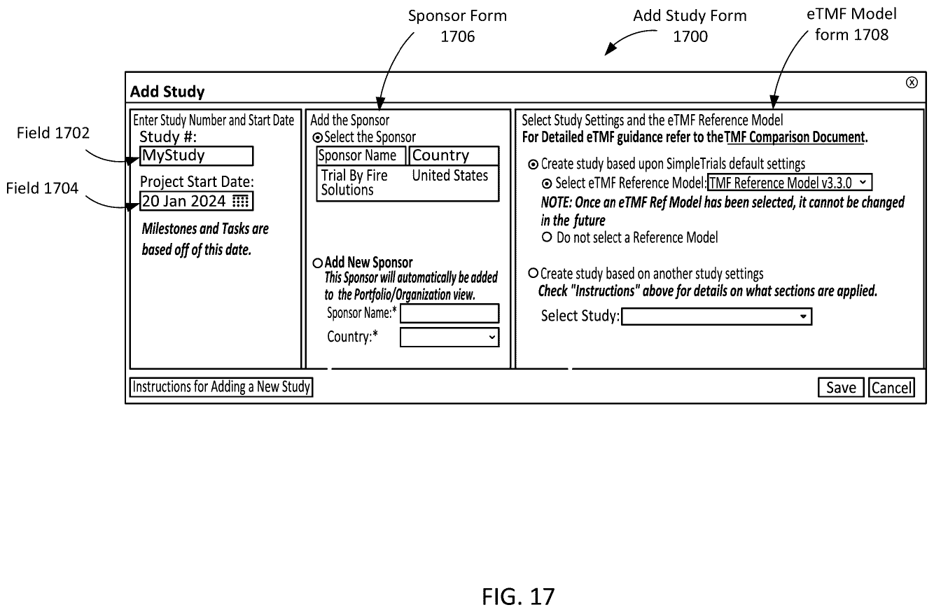

In step 520, the user interface module 322 may provide a form that allows the authorized user to add a study to a portfolio workspace generated in step 516. An add study form 1700 of FIG. 17 may be provided by the user interface module 322. The user may interact with various parts of the add study form 1700 to create a new clinical study workspace. In one example, each study added to a particular portfolio workspace may be associated with one clinical site of a clinical trial. In some embodiments, each study may be associated with one clinical trial with multiple clinical sites associated with the clinical trial.

The user may provide the name of the clinical study in field 1702 and a projected start date of the clinical trial may be provided in field 1704. Milestones and tasks (e.g., defined by the user during configuration) may be generated by the schedule module 318 based on the projected start date. The schedule module 318 may utilize past duration of one or more tasks associated with the clinical trial clinical site and a projected start date to provide an estimate of when particular milestones may be achieved. In some embodiments, milestones include projected dates to provide regulatory documents to the required government regulators. Users may interact with the user interface 1900 to change the projected time line of the clinical trial to account an early completion of one or more tasks or a delay of one or more tasks or some combination therewithin.

The user may interact with a sponsor form 1706 section of the add study form 1700 to add a sponsor to the study. The sponsor form 1706 allows the user to choose a previously submitted sponsor, or to associate the study with a new sponsor. The sponsor form 1706 allows users to add participating countries as sites. For example, if the authorized user is a member of a CRO, the authorized may be responsible for overseeing or managing multiple clinical sites for different sponsors. The sponsor form 1706 allows the authorized member to add multiple studies to a portfolio workspace, where different studies have different sponsors.

The user may interact with a reference model form 1708 to select the reference model. In some embodiments, users may specify whether the eTMF Reference Model is used or if another reference model is used. For example, the user may specify a DIA Reference model. DIA Reference model is another standardized framework developed by the Drug Information Association (DIA) to support the organization, management, and exchange of documentation in the pharmaceutical and clinical research industries. In another example, the user may specify a Clinical Data Interchange Standards Consortium (CDISC) reference model.

Other properties of the study workspace may be required by the authorized user to configure, set up, or add a new study workspace. These properties may include, but are not limited to, Essential Document Definitions, trial site essential document folder and placeholder settings, specify country-specific items, and update requirements for availability at site activation or study end. Once trial sites are added and the country of the sponsor and trial site are designated, the folders may be automatically populated in the Documents area of the study workspace. Different countries may have different regulatory requirements, but the clinical study operations platform 102 is knowledgeable in the different regulatory requirements of different countries.

Users also have the option to provide configurations by adding subject definitions (not shown in FIG. 17), such as participant screen failure reasons and protocol deviation reasons for tracking screen failure reasons. This option may be found in the Screening and Enrollment area and protocol deviations in the Protocol Deviations area of the navigational panel. Another property of the study workspace that can be configured or customized by users of the clinical study operations platform 102 includes participant visit schedules, visit schedules and visit windows for tracking visits within participants of a particular clinical site or clinical trial or geographic area may be added. Furthermore, matters relating to contract and payment actions of the participants of the particular clinical site or clinical trial may also be tracked. Sync options are also available if tracking participant visits and projected participant visits may take place in the visit calendar and subject visit areas of the navigational panel of the study workspace.

In some embodiments, in step 522, the management module 306 may send a request to the portfolio and study workspace datastore 328 to create or update a study workspace entry to generate a study workspace (e.g., a platform for the clinical study). An example study workspace can be found in FIG. 18A. The user interface 1800 of FIG. 18A includes identification information such as the name of the clinical study, sponsor name, the current status and a timestamp associated with when the study was created. Study metrics including the number of countries and sites are also included in the study workspace which gives a summary of a particular clinical trial or particular clinical site. A table of high level milestones can also be found in the user interface 1800.

Additional information regarding the study workspace can also be found in FIG. 18B. An example of a study that utilizes these subject definitions can be found in user interface 1802 of FIG. 18B. The user interface 1802 is a study workspace and includes a screening fail table 1804 listing all the criteria in which clinical trial participants failed the screening and an inclusion or exclusion code associated with the particular criteria. The user interface 1802 also includes a protocol deviation table 1806 which lists protocol deviation categories. Protocol deviation are instances where the conduct of a clinical trial diverges from the approved clinical trial protocol or regulatory requirements.

In one example, in step 524, the management module 306 may generate a management plan associated with the study workspace. The user interface 1900 may enable an authorized user to define major study-related milestones and tasks that may be tracked. In the user interface 1900, tasks with a duration of 1 or more days are indicated by horizontal bars showing progress over time. Milestones have a duration of zero days and are indicated by a shape that represents the date a milestone may be or has been achieved. Milestones and tasks are a two-level project plan, with child subtasks representing individual tasks and milestones line items and parent tasks representing an aggregate of their child subtasks.

As discussed herein, the management module 306 may enable the user to configure their workspace for the clinical study in any number of ways. For example, an authorized user and/or a reference model may further select and provide information from configuration fields, picklists, and trackers. In some examples, the authorized user may utilize the management module 306 to define fields and columns and group them into functional system views. The authorized user may allow the authorized user to configure columns in the scope of all studies associated with the authorized user (e.g., the entity that has one or more subscriptions supporting any number of clinical studies). As a result, the authorized user may view and make changes to the configuration of one, two, or more clinical studies being managed by the clinical study operations platform 102 (e.g., across different platforms, each platform being created for a particular clinical study). The management module 306 may receive and implement authorized user-created tables for trackers. The management module 306 may limit addition of client-configured trackers based on subscription plan limits and/or add-on credits. The management module 306 may enable client-configured trackers in common formats and may import client tracker data based on a worksheet template. In various embodiments, the management module 306 may enable and authorize user to organize and find specific records in custom trackers. The management module 306 may provide the authorized user to make limited changes to system fields such as color and order.

The management module 306 may further allow a user to create definitions of what constitutes a protocol deviation. In one example, the authorized user provides a name and code which the management module 306 uses to generate a deviation category pick list that is available in an interface (e.g., a protocol deviations view).

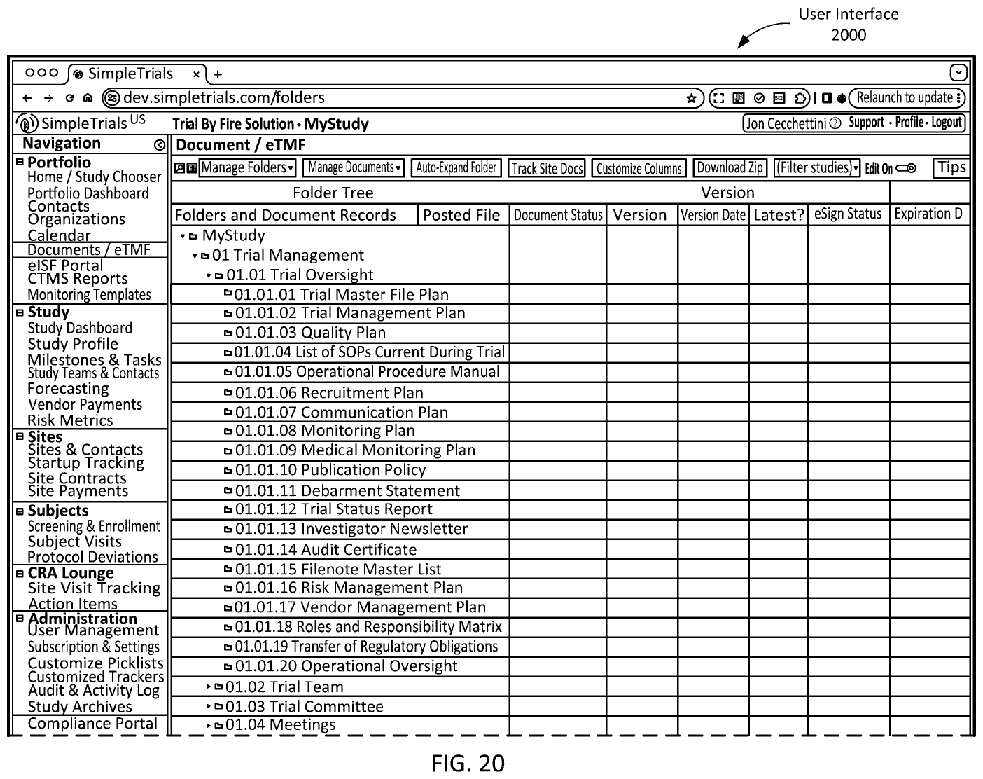

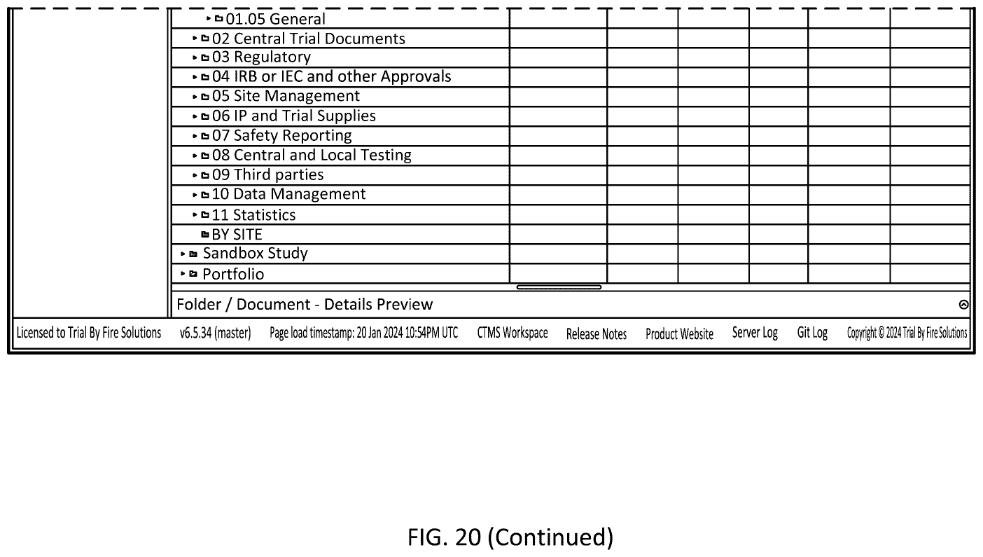

In various embodiments, step 526, the management module 306 may send a request for data required to generate an eTMF reference model for a particular clinical trial or clinical site associated with the study workspace. An example of the eTMF reference model can be found in user interface 2000 of FIG. 20. The user interface 2000 includes a file or document management that tracks all study-related documents. Furthermore, the user interface 2000 allows users to upload files to the file management.

In various embodiments, the management module 306 may provide options to an authorized user to select (e.g., in a pull down menu) one from any number of eTMF reference models. Each eTMF reference model may be associated with a pre-defined template for collection of study-level, country-level, and site-level documents. The authorized user may subsequently make manual changes or additions created in the workspace (e.g., after rules or configurations are performed by a selected eTMF reference model).

Users of the clinical study operations platform 102, whether the user is an authorized user or other invited users, may have clearance to search for documents, view document dashboards, manage document folders (e.g., create, edit, or remove folders), manage documents records (e.g., create, edit, remove, track details), and manage document files. eTMF reference models may be used in clinical trials, while Drug Information Association (DIA) Reference models used to regulate content across the drug lifecycle.

Examples of systems, environments, and/or configurations that may be suitable for use with the digital devices described herein include but are not limited to, personal computer systems, server computer systems, thin clients, thick clients, handheld or laptop devices, multiprocessor systems, microprocessor-based systems, set-top boxes, programmable consumer electronics, network PCs, minicomputer systems, mainframe computer systems, and distributed cloud computing environments that include any of the above systems or devices, and the like. A computer system may be described in the general context of computer system executable instructions, such as program modules, being executed by a computer system. Generally, program modules may include routines, programs, objects, components, logic, data structures, and so on that perform particular tasks or implement particular abstract data types.

FIG. 25 depicts a block diagram of an example digital device 2500 according to some embodiments. The digital device 2500 is shown in the form of a general-purpose computing device. The digital device 2500 includes at least one processor 2502, RAM 2504, communication interface 2506, input/output device 2508, storage 2510, and a system bus 2512 that couples various system components including storage 2510 to the at least one processor 2502. A system, such as a computing system, may be or include one or more of the digital devices 2500.

System bus 2512 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

The digital device 2500 typically includes a variety of computer system readable media, such as computer system readable storage media. Such media may be any available media that is accessible by any of the systems described herein and it includes both volatile and nonvolatile media, removable and non-removable media.

In some embodiments, the at least one processor 2502 is configured to execute executable instructions (for example, programs). In some embodiments, at least one processor 2502 comprises circuitry or any processor capable of processing the executable instructions.

In some embodiments, RAM 2504 stores programs and/or data. In various embodiments, working data is stored within RAM 2504. The data within RAM 2504 may be cleared or ultimately transferred to storage 2510, such as prior to resetting and/or powering down the digital device 2500.

In some embodiments, the digital device 2500 is coupled to a network, such as the communication network 128, via communication interface 2506. Any of the elements of the first entity system 104, second entity system 114, and clinical study operations platform 102 can communicate with one or more networks such as a local area network (LAN), a general wide area network (WAN), and/or a public network (for example, the Internet).

In some embodiments, input/output device 2508 is any device that inputs data (for example, mouse, keyboard, stylus, sensors, etc.) or outputs data (for example, speaker, display, virtual reality headset).

In some embodiments, storage 2510 can include computer system readable media in the form of non-volatile memory, such as read only memory (ROM), programmable read only memory (PROM), solid-state drives (SSD), flash memory, and/or cache memory. Storage 2510 may further include other removable/non-removable, volatile/non-volatile computer system storage media. By way of example only, storage 2510 can be provided for reading from and writing to a non-removable, non-volatile magnetic media. The storage 2510 may include a non-transitory computer-readable medium, or multiple non-transitory computer-readable media, which stores programs or applications for performing functions such as those described herein with reference to, for example, FIG. 3. Although not shown, a magnetic disk drive for reading from and writing to a removable, non-volatile magnetic disk (for example, a “floppy disk”), and an optical disk drive for reading from or writing to a removable, non-volatile optical disk such as a CDROM, DVD-ROM or other optical media can be provided. In such instances, each can be connected to the system bus 2512 by one or more data media interfaces. As will be further depicted and described below, storage 2510 may include at least one program product having a set (for example, at least one) of program modules that are configured to carry out the functions of embodiments of the invention. In some embodiments, RAM 2504 is found within storage 2510.

Programs/utilities, having a set (at least one) of program modules, such as those of the clinical study operations platform 102, may be stored in storage 2510 by way of example, and not limitation, as well as an operating system, one or more application programs, other program modules, and program data. Each of the operating systems, one or more application programs, other program modules, and program data or some combination thereof may include an implementation of a networking environment. Program modules generally carry out the functions and/or methodologies of embodiments of the invention as described herein.

It should be understood that although not shown, other hardware and/or software components could be used in conjunction with the digital device 2500. Examples include, but are not limited to microcode, device drivers, redundant processing units, and external disk drive arrays, RAID systems, tape drives, and data archival storage systems, etc.

Exemplary embodiments are described herein in detail with reference to the accompanying drawings. However, the present disclosure can be implemented in various manners, and thus should not be construed to be limited to the embodiments disclosed herein. On the contrary, those embodiments are provided for the thorough and complete understanding of the present disclosure, and completely conveying the scope of the present disclosure.

It may be appreciated that aspects of one or more embodiments may be embodied as a system, method, or computer program product. Accordingly, aspects (e.g., modules discussed herein) may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), a solid state drive (SSD), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program or data for use by or in connection with an instruction execution system, apparatus, or device.

A transitory computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++, Python, or the like and conventional procedural programming languages, such as the “C” programming language or similar programming languages. The computer program code may execute entirely on any of the systems described herein or on any combination of the systems described herein.

Aspects of the present invention are described below with reference to flowchart illustrations and/or block diagrams of methods, apparatus (systems) and computer program products according to embodiments of the invention. It may be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

While specific examples are described above for illustrative purposes, various equivalent modifications are possible. For example, while processes or blocks are presented in a given order, alternative implementations may perform routines having steps, or employ systems having blocks, in a different order, and some processes or blocks may be deleted, moved, added, subdivided, combined, and/or modified to provide alternative or sub-combinations. Each of these processes or blocks may be implemented in a variety of different ways. Also, while processes or blocks are at times shown as being performed in series, these processes or blocks may instead be performed or implemented concurrently or in parallel or may be performed at different times. Further any specific numbers noted herein are only examples: alternative implementations may employ differing values or ranges.

Throughout this specification, plural instances may implement components, operations, or structures described as a single instance. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. Furthermore, any specific numbers noted herein are only examples: alternative implementations may employ differing values or ranges.

Components may be described or illustrated as “configured to,” “adapted to,” “operative to,” “configurable to,” “adaptable to,” “operable to” and the like. Such description or illustration should be understood to encompass components both in an active state and in an inactive or standby state unless required otherwise by context.

The use of “or” in this disclosure is not intended to be understood as an exclusive “or.” Rather, “or” is to be understood as including “and/or.” For example, the phrase “providing alerts or reports” is intended to be understood as having several meanings: “providing alerts,” “providing reports,” and “providing alerts and reports.”

It may be apparent that various modifications may be made, and other embodiments may be used without departing from the broader scope of the discussion herein. Therefore, these and other variations upon the example embodiments are intended to be covered by the disclosure herein.

1. A method comprising:
receiving, by an on-demand system on a network, a first request to create a first account to manage operations of a first clinical study regarding a first field of study;
creating a first account based on credentials provided by a first entity to manage the operations of the first clinical study;
configuring a first workspace based on a first study configuration information provided by a first user associated with the first entity to manage operations of the first clinical study; and
generating a first dashboard to track and provide alerts associated with the first entity to manage operations of the first clinical study.

2. The method of claim 1, further comprising:
receiving, by the on-demand system on the network, a second request to create a second account to manage operations of a second clinical study regarding a second field of study, the first and second fields of study being different from each other;
creating a second account based on credentials provided by a second entity to manage the operations of the second clinical study;
configuring a second workspace based on second study configuration information provided by a second user associated with the second entity to manage operations of the second clinical study; and
generating a second dashboard to track and provide alerts associated to the second entity to manage operations of the first clinical study, the first user having access to the first dashboard and the first workspace based on first user credentials and the second user having access to the second dashboard and the second workspace based on second user credentials, the first user not having access to the second dashboard and the second user not having access to the first dashboard.

3. The method of claim 2, wherein the on-demand system is a multi-tenant system and the first and second entities are different tenants.

4. The method of claim 3, where the first workspace and data associated with the first clinical study is isolated from the second workspace and data associated with the second clinical study.

5. The method of claim 2, further comprising:
receiving, from the first user, a selection of a first reference model from a list of predefined reference models to configure the first workspace, the first reference model being predefined to identify workflows, definitions, and document relationships to assist in managing the first clinical study; and
receiving, from the second user, a selection of a second reference model from the list of predefined reference models to configure the second workspace, the second reference model being predefined to identify workflows, definitions, and document relationships to assist in managing the second clinical study.

6. The method of claim 3, wherein the on-demand system does not allow customized source code from the first user, the on-demand system enabling configuration of the first workspace based on predefined functions that may be selected and configured by the first user.

7. The method of claim 1, further comprising generating a first sandbox with example data to assist in training at least one user associated with the first entity to configure and manage the first workspace for operations management of the first clinical study.

8. The method of claim 1, wherein an authorized user associated with the first entity configures the first workspace by performing one or more of defining categories and subcategories for documents, outlining metadata fields for indexing and searching, specifying document relationships, specifying workflow processes, and establishing rules to ensure compliance with regulations.

9. The method of claim 1, further comprising:
identifying, by the on-demand system, one or more legal documents required based on a type of the first clinical study and geographic locations indicated by the first user;
providing, by the on-demand system, the one or more legal documents; and
and requiring the one or more legal documents to be effectively signed.

10. The method of claim 1, further comprising: generating a portfolio workspace, the portfolio workspace allowing the first user to manage and organize data across multiple clinical trials from a workspace interface to perform at least one of tracking, managing, or reporting on aspects of the first clinical study or a third clinical study.

11. The method of claim 1, further comprising:
the first user providing a configuration of a third clinical study; and
generating the third clinical study based on a previously created fourth clinical study, the previously created fourth clinical study being previously created by the on-demand system.

12. A non-transitory computer-readable medium comprising executable instructions, the executable instructions being executable by one or more processors to perform a method comprising:
receiving, by an on-demand system on a network, a first request to create a first account to manage operations of a first clinical study regarding a first field of study;
creating a first account based on credentials provided by a first entity to manage the operations of the first clinical study;
configuring a first workspace based on a first study configuration information provided by a first user associated with the first entity to manage operations of the first clinical study; and
generating a first dashboard to track and provide alerts associated with the first entity to manage operations of the first clinical study.

13. The non-transitory computer-readable medium of claim 12, the method further comprising:
receiving, by the on-demand system on the network, a second request to create a second account to manage operations of a second clinical study regarding a second field of study, the first and second fields of study being different from each other;
creating a second account based on credentials provided by a second entity to manage the operations of the second clinical study;
configuring a second workspace based on second study configuration information provided by a second user associated with the second entity to manage operations of the second clinical study; and
generating a second dashboard to track and provide alerts associated to the second entity to manage operations of the first clinical study, the first user having access to the first dashboard and the first workspace based on first user credentials and the second user having access to the second dashboard and the second workspace based on second user credentials, the first user not having access to the second dashboard and the second user not having access to the first dashboard.

14. The non-transitory computer-readable medium of claim 13, the method further comprising:
receiving, from the first user, a selection of a first reference model from a list of predefined reference models to configure the first workspace, the first reference model being predefined to identify workflows, definitions, and document relationships to assist in managing the first clinical study; and
receiving, from the second user, a selection of a second reference model from the list of predefined reference models to configure the second workspace, the second reference model being predefined to identify workflows, definitions, and document relationships to assist in managing the second clinical study.

15. The non-transitory computer-readable medium of claim 13, wherein the on-demand system does not allow customized source code from the first user, the on-demand system enabling configuration of the first workspace based on predefined functions that may be selected and configured by the first user.

16. The non-transitory computer-readable medium of claim 13, the method further comprising generating a first sandbox with example data to assist in training at least one user associated with the first entity to configure and manage a first workspace for operations management of the first clinical study.

17. The non-transitory computer-readable medium of claim 13, wherein an authorized user associated with the first entity configures the first workspace by performing one or more of defining categories and subcategories for documents, outlining metadata fields for indexing and searching, specifying document relationships, specifying workflow processes, and establishing rules to ensure compliance with regulations.

18. A system comprising:
at least one processor; and
memory containing instructions that are executable by the at least one processor to:
receive, by an on-demand system on a network, a first request to create a first account to manage operations of a first clinical study regarding a first field of study;
create a first account based on credentials provided by a first entity to manage the operations of the first clinical study;
configure a first workspace based on a first study configuration information provided by a first user associated with the first entity to manage operations of the first clinical study; and
generate a first dashboard to track and provide alerts associated with the first entity to manage operations of the first clinical study.

19. The system of claim 18, wherein the instructions are further executable by the at least one processor to:
receive, by the on-demand system on the network, a second request to create a second account to manage operations of a second clinical study regarding a second field of study, the first and second fields of study being different from each other;
create a second account based on credentials provided by a second entity to manage the operations of the second clinical study;
configure a second workspace based on second study configuration information provided by a second user associated with the second entity to manage operations of the second clinical study; and
generate a second dashboard to track and provide alerts associated to the second entity to manage operations of the first clinical study, the first user having access to the first dashboard and the first workspace based on first user credentials and the second user having access to the second dashboard and the second workspace based on second user credentials, the first user not having access to the second dashboard and the second user not having access to the first dashboard.

20. The system of claim 19, wherein the on-demand system is a multi-tenant system and the first and second entities are different tenants.
